SEQUENCE LISTING (46 sequences)

SEQ ID NO: 1 - DNA, 6030 bp, Cannabis sativa
gaattcccacaggaatgctttctattaatcaccaaagaattactgaaaatagctcttcgagaggctttctctcctattctaacctttcaagaaggctagatctctcccaagagatttctcaattctaaaccctttcaacataactaaaactgagttctgattccctatgcatacacgatagagaataaagctttcttcatagcaacttaattaaaagctactagccaatacaaaaagaacaattcagcttagaacaattagaacaaagactaaaactgaataaactgctgtcataaaacttatgcttggcatttgtggcttttcctctttcggtagacacatcaagggtttatcaatatccctactaaaatcttaccttgtcctcaagtggaaatcatgcattgatagttgagcaagtctcctgtgtagcttcaaagtcaggtagggagtgccattccatcaaaacgttggaatttgcttgagtaccagctgttataaaatggtgttggaggacctgtccgggttcaacctgtagctccaatttaggtgtcagttgcagacgtaatgtaggagaggagtgagcatcacctactgtgcagcggagctgggagacatggaaaattggatgaataagagctgaattcgatagctggagtctgaaagcgactacaccgattcattgggaatagggtatgggctagcatctagttggcagtttttcattggaccgtttggtgagtgaatgctggttatataatttgatttttaggaagaccttgtctccccaccttaaactcctcttttcgacgttttagatcagtatgggctttcatacgttgctgtgcacgttagagatgcatgatgcattccttaaatcatcacgatctcattctgtgcctgaaaacttttcaatagatgaaaccaccgctgtctcctttttgtaaggcaaaataggtggtagatcttgaccatataatgctttgaaggttgtggacttgagggatgtattatgtgctgtatgaaaaatcagcccaagataaaccatttaggccattgtttgctcatatgtgaaacaatgtagtaggtacgtgtcaagacacctattaagaaccttcaattgcccttcgatggtatggtatgtggtgcttttcttgagttgttttccttgttgcttgaataaatggctccaaaagttgctcacaaaaattcgatcgtgatcggtaatgatggacatggaaattcatgtaagttaacaacttcgcgcataaaagtttcagctagtgtttaagcttccaaaagtgatcatggtcaatctatcaacaaccaccaaaatcgtgtccccaacgttggatttgggaaggccttctatgaatctatgattatttcgtcccaaacttgtgctggaagtgggagggctggagtaatccggtaggttaagtattgaggagtttattttgttgccccaaaaagttcagctgcgatgtgtttataagttttgagcttgcctaaatgtcctcgccatggacttttttaaaatcagctaggatgtgtggaattatggtgggttttctttgggatagctacttttgtagtagaggatgtgttatggagagtgaaaacgtagtgttgttgattactgacgtgaatatcatatcatagctgagtaaggaaggcatcagcggaattaatactagggtaattttagtgggggtaatttggtcaagtaaaaaattcattacatgaaattcgggtttcccaatcataatcccaaagctggtgcaactcagtgagtaaggaaggcatcagcggaattaatgctagggtaattttagtgggggtaatttggtcaagtgaaaaattcattacatgaaattcgggtttcccaatcataatcccaaagctggtgcaactcagctttggctcttttttttctgataaaaaaaaagacgcttaaagacagctttggcttttggcttctagctgcaaaagaagcttaaattaattagagcaatttgcggcgaaacctccttatgttttgatttttatacacttaacccccttatctttatttttggtggcaaaacccccttatgttttaatttttatacacttaacccccttatcttttttttggtggcaaaccccttatgttttaatttttatacacttaacccctctatcttttgttttggcggcaaaacccttcagtttacttttctttgcaaatttaaagttttagttaaatattaccgttaaatactaactagattactactgtatcgacttcgaggttttaccgttacatatacaaaggtgtatacagtggtcatccagttgatatttaacggtaatatttaactggcgtgtcaaatttggaaagaaaaataaacataagggggttttgccaccaaaaaaaagataagggggttaagtgtataaaaattaaaacataagggagttttgccacaaaaaaaaaagataagggggttaagtgtataaaaattaaaacataagggggttttgccaccaaaaataaagataagggggttaagtgtataaaaatcaaaacataagggggtttcgccgcaaattgctcaattaattaataagccacttttttttgttttaccaaacagtttggcatttagcttattcaacaagtgcttttaagtttctctaaaagctgtctaaatggggcctagatctctccaaagagatctctttcaaatctaaactctttcaacataaccgaaactgagttctaattctctatatatagaataaagcttgcgcatcaacaacttaattaaaagctactaactaatacaaaaagaacaactcagcctaaaacaattaatgaaagactaaaactgaataaactgctttaataaaacttatgcttggccttttctgttttttgtagacacattggtccagggttatcagaaagcagtctgagcaagtgatctggttgtatttgttttttgttttacaagttctgtgactcttgttgatgcaattttttttatatataatcaactaagttgcctgcattctagaaaaactggtgcatctcaaggtttttgcactatgtttttgccttattgtcatatgtttgaacttgcaggttttggaagagattcgggttggcctggaattaaatgactatggtatgcagcagagacgcttagcccatatgcgatttttaggagagctatacaactatgagcatgtggattcgtctgttatttttgagacgctgtatttgattcttgtctttggccatggctcaccagaggtattcttctatgtgctcagaacaattgttatgaatcttttttgtttctgacatacatgctttgatgtgaaaataatagatggaatctcttcataccaacggggtttttttcttttaagttttatttatagatgctatttgggatggtgatacagctaaagtgcgatatggaaaatgatttgtgtcatttggggtgtcatatagctaaaaggagaacatggtcttatggacttgtatgtcaaaaatgaatttttaatgacactatgccatgtataactatgtctttgtttttcttaatatttattttgtttgatttttggtgcgaaatatattatgtaaaaatattctgacatggtatctaatattattttcccatctcatagcaagatctactagacccaccggaggattgtttccgcatgaggatggttattactctccttgagacctgtgggcactactttgatcgaggctcttctaaaaggaaacttgatcgatttttaatacactttcaaagatatatcttgagcaaaggtgctttaccactggatattgaatttgatttacaggtatttattgcagtttatttttttaatttcatttgtagttgttctttttgcatgctttgatgactggaagtaatattatagcaccgtttatattacacctatattatatgcttgaaatgagaaaacactgaagtctgttatatcccttatgtttttgaaaatgtgttgtatcttcattccctttatgcactcaatttattaccttcctggaatttgagggagatctattgtactgaagctttcattttcaggatttatttgctgatttacgtcccaatatgactcgatactcatctattgaagaagttactttagctctggtggaacttgaggagcatgaacgtactctcccatctgacaaaacaagtagtgagaagcactctgacagtgaaataaggagttctttcaactctatttcagcaaatggacagagtgctgtgaatggtaacgagggaaatggtaggttgcatgatggccttggagacagtgacagtgattctggaagcgggactcttgatcaagaggggcgtgatgaagaagaactggatgatgagaatcatgatgaagaatgtgatactgatgaggaggatgacgatgggggtggacctgcctcagatgaggatgaagtccacgtgaggcagaaggtgatggaagttgaccctcttgaagcagcaacttttgagcaagaactaaaggctgtcatgcaggcaagtgttttttctttaactcttgtggcatatcatttaatgtgttttattctgatgactgtggtgtggggcttgtgtgctggtggtgaatattgattatttctaacgtcaaattaaattaaatgcaggaaagcatggatcagcgcaggcaagaactacgtggtcgacccacattaaatatgatgataccaatgaacgtgtttgaaggatcaaccaggggggttggtggtgaaagtggtgatgaagcattggatgaggagggtggaggaattaaggacgtccaggtaaaagttcttgtgaagcgtgggagcaagcaacagactaggcagatgtacatccctcgggattgctctcttgtacagagcacaaaacagaaagaagcagctgagcttgaggagaaacaagacatcaaaaggctagttttagaatataatgatagagaggaggaggaactgaatgggttgggaacccaaacattgaaccatatgcagggtagcggcagtagaggttccacccgaggtcacttgtgggaaggaagcagtgggagggggggaacacgtcatcggcattattccggtggtggaatcttctacaacagaaaaaagtgaaggttattatgctagaaggctttcttgtttttgccacaagttcaaataggtacagtaaactatctcaatttctaacgagatttgtcgaggctgcctaaaacgaagcggtgccttttctgcaaggctgatgtgccgtttcttaccccattctgatgagctcaatagtctaaaaaaagtagcgcttcgggaaaaatgttttaagaagttcaattgccaggtagttagtgcgagacacaatgctatgtgttttcagtttctgatctgcaatgtacctgtgggatttgattgtgtgctgtatagatcctgcgtttggcatttgctgcagtttgcatgtatatatattatgtaagacaaaatgaagatagtatgaccaaaaatctcttgctttgttcttattttagatatagtgtaatgatttctgttatacttgtgtagtgactgactcattcaaaccgaacttaatatgcatttagaatggttgagagtttatgataatgataatgcaataccaatttcatatgaccaattcctctattgtatccatattgttttggacagttaatgtgttgtaatggtactcatcgttttagttgccccacggctacaaatgttttcaaatcgtttcgagtccaattgggtaaggtgtggtaattaaaacaaatctataatgataatgtaataccaaccttatacgaccaattcttgtatggtatccatattgttttggacagttgttttttttttttcttgtaatggtactcaccgtttcagttgtccaatgttttcaaatcgtgttttgagtccaattggaaaaggtgtggtagttttaataaatctatatggagttt

SEQ ID NO: 2 - DNA, 171 bp, Cannabis sativa
aatgtgttgtaatggtactcatcgttttagttgccccacggctacaaatgttttcaaatcgttcgagttccaattgggtaaggtgtggtaattaaaacaaatctataatgataatgtaataccaacctatacgaccaattcttgtatggtatccatattgttttgacagtt

SEQ ID NO: 3 - DNA, 24 bp, Cannabis sativa
gtacagtaaactatctcaatttct

SEQ ID NO: 4 - DNA, 24 bp, Cannabis sativa
accacaccttttccaattggactc

SEQ ID NO: 5 - DNA, 210 bp, Cannabis sativa
gctactggaaacctgtctattttctagtgcaatgaagtataatatggacaatgaggcaaggcaatgtaagtaggttaagagataaaagtttcaaggcactggaaaaggaatggaaagtataatcatagttaggcagagaaagatttgaactgttatacggttcttttatgggatgggagtttattatgaatttcaagtggagaaaaaaaa

SEQ ID NO: 6 - DNA, 201 bp, Cannabis sativa
aacactcaacacacaaatatacaaatatcaaatacacatacactcacacacacaaatggtaatgaaagaaagaagacggtaccttaaaagcagaacgagcgacatagcccaggcgttgagcttctctgtagaaaaaatctggagctccagctccactcatctctgcaaatgttaaaaccctaaaccccggtagtcgcacag

SEQ ID NO: 7 - DNA, 201 bp, Cannabis sativa
ctggcggtgcccaaaattttgggcacccggttgaaaatttgattgcctggttgggaactttttcgagtatctcaatttttgtgatatttaggatattgactcttcatttatggatggaaaaattttaaattataaatttaagttgtaaaaagtattttagtgagacatataccaatttaccattaaatattttctactact

SEQ ID NO: 8 - DNA, 201 bp, Cannabis sativa
gttatatggagacttcacatccaccactaacaacgtggccatgatggtggtttaccttgggctagtgctgctagtgagtggcagtttgatcttgccacatggagtaatggcagctccactgaaaccatacattagatgttcgcatggatttaggtgtttgagtgacaaaccaatctggattaaagtttgcttgaataagat

SEQ ID NO: 9 - DNA, 201 bp, Cannabis sativa
cctccttgtttttacataatcaatgcatgccttattcttcttttactttatctttatttttttttctttaaaattacattttctttagtttattggtgatatcccttctagcttagcataattttgcctgtactattaaggcaatcaaattttgtcttcatttttacaattttagtattttcttgtactgaataagatttt

SEQ ID NO: 10 - DNA, 201 bp, Cannabis sativa
gtgtctccctaaatcccgtgggagccttggcttccgaaaaactaagaaaatgaatcaggatttcttggctaagtaggcttggaaactgcttactaaatcccaatctttgtgttgaagaattctaagagctaaataccttagaggaaaggatatgcttaagtgtaaggctaagtctgttgacttttggttttagaaaagtgt

SEQ ID NO: 11 - DNA, 201 bp, Cannabis sativa
ccttcagattataatacaaaacacaataagaaatttgaaaatacagataaagaagcaatatataaccatgtggaactgttcaagtatgtctacgcaatcaacattaccggatttccatcagaatctccaaagccttttttgggaccatattaatagattgcaactgaaaatttccatgtcaacattagtagaagaagatca

SEQ ID NO: 12 - DNA, 201 bp, Cannabis sativa
tttaaatgagtaatttaatttcatgttattatacgtaaaaaaaaatatatgtatatatatgaaaattaacattaaaattttactttttatttttggattagatttatccaattcaatccaataaatactagatctaaattattggatgaatctaattcgatccataaatattaggtgtaaaatattagataaacttgaatt

SEQ ID NO: 13 - DNA, 201 bp, Cannabis sativa
actggtttaatatataaatatataaaataatataaaatttaaaagaagaagaatataaagagagaaaaaaattaaatgaaaatttttcagttagagtgaattcttatttatacaaaaatatgattaaaagaatgaaaaattaaactaatgtaataaagaaaaatacaactaagcattaatggcgataattaatttggtgat

SEQ ID NO: 14 - DNA, 201 bp, Cannabis sativa
ctaagtttgagatgaaggacttaggaaatgccacttaaatccttggaataattattgtgagggataaaggcaagggatcctaaagatgtgtcaggaagataacattcagaaggtgattgagaagtgttgggaatattttactaggatctagatttactaacaagtatgatgattaacatcctaaatatgaatatctctaaa

SEQ ID NO: 15 - DNA, 373 bp, Cannabis sativa
aatatgcatttagaatggttgagagtttatgataatgataatgcaataccaatttcatatgaccaattcctctattgtatccatattgttttggacagttaatgtgttgtaatggtactcatcgttttagttgccccacggctacaaatgttttcaaatcgtttcgagtccaattgggtaaggtgtggtaattaaaacaaatctataatgataatgtaataccaaccttatacgaccaattcttgtatggtatccatattgttttggacagttgttttttttttttcttgtaatggtactcaccgtttcagttgtccaatgttttcaaatcgtgttttgagtccaattggaaaaggtgtggtagttttaataa

SEQ ID NO: 16 - DNA, 201 bp, Cannabis sativa
attgattataaaataaatagagcttaagaaacaaaaaaattaaagaagaagaaaactaagatttttacgtggttggagcgttgatgaactttagtccacgagtccatatattattaatttgagaaactttgatgttttacacaaggataatattttttccaaacttaagaaccctaatttaagtcttagtatttgaataat

SEQ ID NO: 17 - DNA, 201 bp, Cannabis sativa
ataaatagagcttaagaaacaaaaaaattaaagaagaagaaaactaagatttttacgtggttggagcgttgatgaactttagtccacgagtccatatattattaatttgagaaactttgatgttttacacaaggataatattttttccaaacttaagaaccctaatttaagtcttagtatttgaataatgattttccacca

SEQ ID NO: 18 - DNA, 201 bp, Cannabis sativa
aaaattaaagaagaagaaaactaagatttttacgtggttggagcgttgatgaactttagtccacgagtccatatattattaatttgagaaactttgatgttttacacaaggataatattttttccaaacttaagaaccctaatttaagtcttagtatttgaataatgattttccaccatttgcatgaagatacaatcttct

SEQ ID NO: 19 - DNA, 201 bp, Cannabis sativa
aagatttttacgtggttggagcgttgatgaactttagtccacgagtccatatattattaatttgagaaactttgatgttttacacaaggataatattttttccaaacttaagaaccctaatttaagtcttagtatttgaataatgattttccaccatttgcatgaagatacaatcttctattataggctagggttaagaca

SEQ ID NO: 20 - DNA, 201 bp, Cannabis sativa
ggttggagcgttgatgaactttagtccacgagtccatatattattaatttgagaaactttgatgttttacacaaggataatattttttccaaacttaagaaccctaatttaagtcttagtatttgaataatgattttccaccatttgcatgaagatacaatcttctattataggctagggttaagacagttaggatttttg

SEQ ID NO: 21 - DNA, 201 bp, Cannabis sativa
gttttacacaaggataatattttttccaaacttaagaaccctaatttaagtcttagtatttgaataatgattttccaccatttgcatgaagatacaatcttctattataggctagggttaagacagttaggatttttgtatgaagctaaacccactgaaggagtaaatacatttaatagtgactttagattaattagaaaa

SEQ ID NO: 22 - DNA, 201 bp, Cannabis sativa
ttacacaaggataatattttttccaaacttaagaaccctaatttaagtcttagtatttgaataatgattttccaccatttgcatgaagatacaatcttctattataggctagggttaagacagttaggatttttgtatgaagctaaacccactgaaggagtaaatacatttaatagtgactttagattaattagaaaagaa

SEQ ID NO: 23 - DNA, 201 bp, Cannabis sativa
acttaagaaccctaatttaagtcttagtatttgaataatgattttccaccatttgcatgaagatacaatcttctattataggctagggttaagacagttaggatttttgtatgaagctaaacccactgaaggagtaaatacatttaatagtgactttagattaattagaaaagaactcatttacaagataacgatcatcta

SEQ ID NO: 24 - DNA, 201 bp, Cannabis sativa
cttaagaaccctaatttaagtcttagtatttgaataatgattttccaccatttgcatgaagatacaatcttctattataggctagggttaagacagttaggatttttgtatgaagctaaacccactgaaggagtaaatacatttaatagtgactttagattaattagaaaagaactcatttacaagataacgatcatctat

SEQ ID NO: 25 - DNA, 201 bp, Cannabis sativa
catttggttattatatatatattcatgatttttttttttctatttattactattgacaatgttgttattttagttatttcaaacttttaattgcattacacttgtaaggataatatttttatggagtattaaatataaataaagagtgatatttaatttttatgtatttttcttcatatttggaaattaatactaaaaatt

SEQ ID NO: 26 - DNA, 201 bp, Cannabis sativa
acttgttataaattatagttgaacttctttatattgttattgctcaaatagaagttaacaattatacaagtcttatacattatgggttcagaaactttgattgaagtgatattggctatactccttcctccagtgggtgttttcctccgctttggttgtgcggtaataattaatcttaaatatcttattatgtctatgcca

SEQ ID NO: 27 - DNA, 201 bp, Cannabis sativa
caaggtctgctcaattccttgatcatcaagcaatctcaattccttgatcatcaagcaatttatcagtagtgttgttagtgggtttgctattagctttttggtaaaaaaaaaaaaaaaaaaaaaaaaaaaacgagtgttcgagtctcctaatttgagccaaaaaacacgagaccgttgttggcaataatcctcttcttaagc

SEQ ID NO: 28 - DNA, 201 bp, Cannabis sativa
aagaaggaggtttcggcatcaaagattcagagaaagagatccaggttcagatattgataatgctctgctacagaaaggaatcaagggctagatatctgaacggaaggagtcattatattccgctgcaaccaatgtaaggtttcctaaactttatatgtgtttatttcatcgttttagaaagttcatatttagggtgttaat

SEQ ID NO: 29 - DNA, 201 bp, Cannabis sativa
tttttctcccatctgattcaaatgtaatattttgattgaaattcactcaaataagaggaagacagagaaggagtgaaaagaccaagattggcagagttattataccaaacaagaaaagagcaaaatactaacatgtaaggatattaagacaaagacaatttagtgatgaaacttacagaagctctgaacttatcaatctct

SEQ ID NO: 30 - DNA, 201 bp, Cannabis sativa
ttgacacaacacgaaactggcacgagcacaattagacacgaaaaaatatgggcattagcacgacataggcacgacaaaagatgaaaaatatgtccttaagtacaatacgacacgagaagtagggctgaacatttaaacccgcaaacccgaaaacccggcaacccaccccgacccgctgccgaaaaaaaaccgaaaaattga

SEQ ID NO: 31 - DNA, 201 bp, Cannabis sativa
acctttgaaagggatcctttcattttgaacatgaacttattgtctcttataccgtcaagaatttaattaattctccataataaaaaagtattgaatgagtggtttggtggtaaaatgcttactctcaactagttttttaagccgttaaatataagaaaaatgtctcgcagcttagccaccctagacatccattttaggcac

SEQ ID NO: 32 - DNA, 201 bp, Cannabis sativa
tttgattgtttttttaaaataagataaaaagataaaatagaaatatttatgaaaataaagaattatataagtgaaaatgttttagacaaaacgtaaaaattaaaaaaaaaaaaaaaaaaaaaaaaaaaaaaaaaaggagtacaacggtttatgcaaacgtgtataattaacctaataattataataagaatagaattagga

SEQ ID NO: 33 - DNA, 201 bp, Cannabis sativa
aagaagttgaacctcctcatgagacaaagacaaatagtagttctttgactgatcttcaaagaagcattataaaagaattattagaaatcatatcgtctcctattgaaaataaacaacaacaagatcaatctaatgaagttgataaaataattattcaagatgatggcataggcctcgatgatctccctgaggaattcttca

SEQ ID NO: 34 - DNA, 201 bp, Cannabis sativa
attttttatgaatcttaaattcaaattctttttttaatgttttatatggttttttttttttttttagtttaattggttaaataatgtcatatttagtggcatttactttttatgtcatatttatcataaccttaataattttttccatattttttaaaatagcccatcaaattttgtccccgaatgttaagcggggcggga

SEQ ID NO: 35 - DNA, 201 bp, Cannabis sativa
aataataaataaaaactttaacaaaaaaaattaattaaaaaattattcgaactttaaattaattttaataaactaaccaatttcatgagttactaataatgttttattaatttatatttaaaagttaatcttgtatctttgctaaacttgtattatagagcaattttaatagatttttaaaattatttataaaagtattaa

SEQ ID NO: 36 - DNA, 201 bp, Cannabis sativa
aaataattattatttagttttgataataaagttattggtaagatttcatatatatacatattattaataactttttatatttgattataatttattaatttcttaaatattaattaacatgaaaaaattactaaataaagtaacataaatatatcttaaattagcaatcaatttcatgggttacttaaaattatttttatt

SEQ ID NO: 37 - DNA, 201 bp, Cannabis sativa
taattaaatttcttaatttgtaactaatctataagaagtcatgaaaagaaaaaatattttaagaaaattggtaagcaaatttataattcatataataataatagattgatgtaaattatagagaaaagtaaaactcaataccaaattacaataaatcttaaattataaatgttagatcgtaagtagaccaaacaaaaaaaa

SEQ ID NO: 38 - DNA, 201 bp, Cannabis sativa
acataaattaattaataactatatactgattgaataaagaggataagaaaaaacttatcacatcaaaatgatctccaaatcttcattatttttaaatatcccatttgtttcgaacattgttatatatctactttgttccacctacaccacttagacgaaacccaatgcagtttgctaacgttaaagattagccaatttagt

SEQ ID NO: 39 - DNA, 201 bp, Cannabis sativa
tgattgaataaagaggataagaaaaaacttatcacatcaaaatgatctccaaatcttcattatttttaaatatcccatttgtttcgaacattgttatatatctactttgttccacctacaccacttagacgaaacccaatgcagtttgctaacgttaaagattagccaatttagtttacattttcattacattttgctcta

SEQ ID NO: 40 - DNA, 201 bp, Cannabis sativa
tgattattcggattgaaacaatagataatttatagcgtatctattcttggtgaatagagtattttatgtaatcaggagtgcaatttcgaatctatagttgagtgaggaggaattaataataaagaaaatttacttgataaattctagaattacttattgagtgcttgattatataggcccatgtccttgtactagttgaga

SEQ ID NO: 41 - DNA, 201 bp, Cannabis sativa
ggaattaataataaagaaaatttacttgataaattctagaattacttattgagtgcttgattatataggcccatgtccttgtactagttgagataataatgtcttgtagactcaattaattaattttaattaatcaattagaattctatttatgagtttcactaagtaagggcttatttgagaagaaaatgaggatttaag

SEQ ID NO: 42 - DNA, 201 bp, Cannabis sativa
aaggtacaatctatgctactatattttaaaatattcattatacattgtaaatattgaaatatgtaaattttttacttatatttcaggttgaaagtatatatgtttatatatgcacacacgtctataatgaaaggtttttgttttaatttcaatttcaagaataaaaattacagtaatttaaattgaattcatatctttctt

SEQ ID NO: 43 - DNA, 201 bp, Cannabis sativa
atctatgctactatattttaaaatattcattatacattgtaaatattgaaatatgtaaattttttacttatatttcaggttgaaagtatatatgtttatatatgcacacacgtctataatgaaaggtttttgttttaatttcaatttcaagaataaaaattacagtaatttaaattgaattcatatctttcttttaattac

SEQ ID NO: 44 - DNA, 201 bp, Cannabis sativa
catattttaatagtggtatatataattttgttagcatgatatatattttttgagtattaatttagtattgttataatttttttgtggtatataattttatcactatagtatattaattttcagtaatacgatatatcaaatactgctgtatatatttaatgatctaatattttattcattttgttacaatataataatatg

SEQ ID NO: 45 - DNA, 22 bp, Cannabis sativa
ctaaaaaaagtagcgcttcggg

SEQ ID NO: 46 - DNA, 24 bp, Cannabis sativa
tgtagccgtggggcaactaaaacg

(19) United States
(12) Patent Application Publication
(10) Pub. No.: US 2022/0333214 A1
(43) Pub. Date: Oct. 20, 2022

(54) METHODS OF DETERMINING SENSITIVITY TO PHOTOPERIOD IN CANNABIS

(71) Applicant: 1769474 ALBERTA LTD., Edmonton (CA)

(72) Inventors: Gregory Baute, Comox (CA); Shumin Wang, Vancouver (CA); Jason Sawler, Centreville (CA); Jose Celedon, Vancouver (CA)

(73) Assignee: 1769474 ALBERTA LTD., Edmonton, AB (CA)

(21) Appl. No.: 17/439,757

(22) PCT Filed: Mar. 31, 2021
(86) PCT No.: PCT/CA2021/050442

§ 371 Date: Sep. 15, 2021

(30) Foreign Application Priority Data
Apr. 1, 2020 (CA) 3077823

(51) Int. Cl. C12Q 1/6895

(52) U.S. Cl. CPC C12Q 1/6895; C12Q 2600/13; C12Q 2600/156

(57) ABSTRACT

This disclosure pertains to markers and methods useful in identifying Cannabis plants that have a day length neutral phenotype, and methods of breeding plants having a day length neutral phenotype.

### CROSS-REFERENCE TO RELATED APPLICATIONS

This application claims priority to Canadian Patent Application No. 3077823 filed Apr. 1, 2020. This patent application is herein incorporated by reference in its entirety, including without limitation, the specification, claims, and abstract, as well as any figures, tables, or drawings thereof.

### FIELD OF THE DISCLOSURE

This disclosure relates to the field of genetic markers for an day length neutral phenotype in Cannabis. More specifically, this disclosure relates to methods and compositions useful for identifying individual plants that display a day length neutral phenotype, or are a carrier for that trait.

### BACKGROUND OF THE DISCLOSURE

Much of the Cannabis in production today is day length (photoperiod) sensitive, meaning that it initiates flowering only after a transition from long photoperiod days to short photoperiod days. This has several limitations, especially at more northern (and more southern) latitudes.

### SUMMARY OF THE INVENTION

The present disclosure pertains to an about 20 megabase region containing about 1,172 genes, any of which could be a causative gene responsible for the day length neutral phenotype in Cannabis. For example, the present disclosure pertains to the utility of an allele at a polymorphic site in an about 20 megabase region located in chromosome CM010796.2 between about 40 megabases and about 60 megabases, and alleles of additional polymorphic sites in linkage disequilibrium with this allele, for identifying Cannabis plants that have a day length neutral (i.e. have an “autoflowering”) phenotype. In some embodiments, the region is any one of about 1 megabase, about 2.5 megabases, about 5 megabases, about 7.5 megabases, about 10 megabases, about 12.5 megabases, about 15 megabases, about 17.5 megabases, or about 20 megabases located in chromosome CM010796.2 between about 40 megabases and about 60 megabases and the disclosure pertains to the utility of an allele at a polymorphic site in any of these regions, and alleles of additional polymorphic sites in linkage disequilibrium with this allele, for identifying Cannabis plants that have a day length neutral (i.e. have an “autoflowering”) phenotype. For example, the Cannabis UPF2 gene is within an about 1 megabase region and contains 46 additional genes and numerous mutations, any of which could be the causative allele responsible for the day length neutral phenotype.

Various aspects of the disclosure relate to the utility of an allele at a polymorphic site within an about 20 megabase region in Cannabis chromosome CM010796.2 located between about 40 megabases to about 60 megabases and alleles of additional polymorphic sites in linkage disequilibrium with the allele, for identifying Cannabis plants that have a day length neutral (i.e. have an “autoflowering”) phenotype. In some embodiments, the polymorphic site is in the Cannabis UPF2 gene, and alleles of additional polymorphic sites in linkage disequilibrium with this allele in UPF2, for identifying Cannabis plants that have a day length neutral (i.e. have an “autoflowering”) phenotype.

Various aspects of the disclosure relate to a method comprising testing nucleic acid from a Cannabis plant to determine the presence or absence of a variation in a polymorphic site within an about 20 megabase region in Cannabis chromosome CM010796.2 located between about 40 megabases to about 60 megabases. In some embodiments, the disclosure relates to a method comprising testing nucleic acid from a Cannabis to determine the presence or absence of a variation in a polymorphic site within the endogenous UPF2 gene. The methods are for identifying whether or not the Cannabis plant has a day length neutral phenotype, or is a carrier of a trait for the day length neutral phenotype. The presence of the variation indicates that the plant has the day length neutral phenotype or is a carrier of the trait for the day length neutral phenotype.

Various aspects of the disclosure relate to a method comprising testing nucleic acid from a Cannabis plant to determine the presence or absence of an allele that is in linkage disequilibrium r²=0.9 to 1 with a variation in a polymorphic site within an about 20 megabase region in Cannabis chromosome CM010796.2 located between about 40 megabases to about 60 megabases. For example, various aspects of the disclosure relate to a method comprising testing nucleic acid from a Cannabis plant to determine the presence or absence of an allele that is in linkage disequilibrium variation r²=0.9 to 1 with a variation in the endogenous UPF2 gene.

Various aspects of the disclosure relate to a method comprising testing nucleic acid from a Cannabis plant to determine the presence or absence of an allele at a polymorphic site as indicated for any one or more of the example nucleotide sequences identified in Table 1, 2, 3, or 4.

Various aspects of the disclosure relate to a method comprising testing nucleic acid from a Cannabis plant to determine the presence or absence of a first allele that is in linkage disequilibrium r²=0.9 to 1 with a second allele at a polymorphic site as indicated for any one or more of the example nucleotide sequences identified in Table 1, 2, 3, or 4.

Various aspects of the disclosure relate to a method of producing a Cannabis plant with a day length neutral phenotype. The method comprises performing a first cross of a first parent having at least one allele associated with a day length neutral phenotype at a polymorphic site within an about 20 megabase region in Cannabis chromosome CM010796.2 located between about 40 megabases to about 60 megabases (for example, without limitation, any one of the example nucleotide sequences identified in Table 1, 2, 3, or 4) with a second parent that has the day length sensitive phenotype. The method further comprises identifying a first progeny plant from the first cross that has the day length neutral phenotype, or that is a carrier of a trait for the day length neutral phenotype, according to the methods described herein.

Various aspects of the disclosure relate to a method of producing a Cannabis plant with a day length neutral phenotype. The method comprising performing a first cross of a first parent that has the day length neutral phenotype and is homozygous for at least one allele associated with the day length neutral phenotype at a polymorphic site within an about 20 megabase region in Cannabis chromosome CM010796.2 located between about 40 megabases to about 60 megabases (for example, without limitation, any one of the nucleotide sequences identified in Table 1, 2, 3, or 4) with a second parent that has the day length sensitive phenotype to produce F₁ progeny. The method further comprises selfing the F₁ progeny to produce F₂ progeny. The method further comprises identifying an F₂ progeny plant that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises identifying a first parent homozygous for at least one allele associated with the day length neutral phenotype at a polymorphic site within an about 20 megabase region in Cannabis chromosome CM010796.2 located between about 40 megabases to about 60 megabases (for example, without limitation, any one of the nucleotide sequences identified in Table 1, 2, 3, or 4) according to the methods described herein. The method further comprises crossing the first parent with a second parent that has the day length sensitive phenotype to produce F₁. The method further comprises selfing the F₁ progeny to produce F₂ progeny. The method further comprises identifying an F₂ progeny plant that has the day length neutral phenotype.

Various aspects of the disclosure relate to a method of producing a Cannabis plant with a day length neutral phenotype. The method comprises performing a first cross of a first parent identified having at least one loss or gain of function mutation allele of one or more endogenous genes (for example, without limitation, the UPF2 gene) within an about 20 megabase region in Cannabis chromosome CM010796.2 located between about 40 megabases to about 60 megabases according to the methods described herein with a second parent that has the day length sensitive phenotype. The method further comprises performing a second cross of a first progeny of the first cross with a second progeny of the first cross to produce a plant that is homozygous for the loss or gain of function mutation allele.

Various aspects of the disclosure relate to a method for producing a day length neutral Cannabis plant. The method comprises decreasing the expression of one or more endogenous genes within an about 20 megabase region in Cannabis chromosome CM010796.2 located between about 40 megabases to about 60 megabases (for example, without limitation, an endogenous UPF2 gene) in the plant.

Various aspects of the disclosure relate to a method of method of generating a Cannabis plant having a day length neutral phenotype. The method comprises i) using a molecular methodology to identify a first plant as comprising a loss or gain of function allele in one or more endogenous genes within an about 20 megabase region in Cannabis chromosome CM010796.2 located between about 40 megabases to about 60 megabases (for example, without limitation, the UPF2 gene); ii) performing a first cross of said first plant to a second plant; iii) performing a second cross of progeny from the first cross; and iv) screening progeny of the second cross for a plant that is homozygous for the loss or gain of function allele in the endogenous gene.

Various aspects of the disclosure relate to a Cannabis plant or plant cell generated according to the methods described herein, as well as seed, plant material, or dried flower of such plants.

Various aspects of the disclosure relate to a genetically modified Cannabis plant or plant cell having a day length neutral phenotype, as well as seed, plant material, or dried flower of such plants. The Cannabis plant or plant cell is genetically modified to have reduced expression of one or more endogenous genes within an about 20 megabase region in Cannabis chromosome CM010796.2 located between about 40 megabases to about 60 megabases (for example, without limitation, the UPF2 gene).

Various aspects of the disclosure relate to an allele-specific polynucleotide for use in the methods described herein.

Various aspects of the disclosure relate to a kit for use in the methods defined herein. The kit comprises at least one allele-specific polynucleotide as descried herein and at least one further component, wherein the at least one further component is a buffer, deoxynucleotide triphosphates (dNTPs), an amplification primer pair, an enzyme, or any combination thereof.

Various aspects of the disclosure relate to a cannabinoids and/or produced by a plant or plant cell as described herein.

Various aspects of the disclosure relate to cannabinoids and/or terpenes extracted or isolated from dried flower as described herein.

Various aspects of the disclosure relate to extracts, concentrates, isolates, or oils of a Cannabis plant, plant cell, dried flower, plant material, or seed as described herein. The extracts, concentrates, isolates, and oils comprise cannabinoids and/or terpenes.

Various aspects of the disclosure relate to an expression vector for generating a day length neutral Cannabis plant, the expression vector comprising a nucleic acid comprising a portion of SEQ ID NO. 1.

Various aspects of the disclosure relate to use of a polynucleotide molecule having a sequence comprising a portion of SEQ ID NO: 1 for generating a Cannabis plant with a day length neutral phenotype.

Various aspects of the disclosure relate to use of a plant or plant cell as described herein for the production of cannabinoids and/or terpenes.

Various aspects of the disclosure relate to a food item comprising a Cannabis plant or plant cell, seed, plant material, or dried flower as described herein.

Various aspects of the disclosure relate to a food item comprising extracts, concentrates, isolates, or oils of a Cannabis plant, plant cell, dried flower, plant material, or seed as described herein. The food item comprises cannabinoids and/or terpenes.

Various aspects of the disclosure relate to a topical comprising extracts, concentrates, isolates, or oils of a Cannabis plant, plant cell, dried flower, plant material, or seed as described herein. The topical comprises cannabinoids and/or terpenes.

Various aspects of the disclosure relate to a pharmaceutical composition comprising extracts, concentrates, isolates, or oils of a Cannabis plant, plant cell, dried flower, plant material, or seed as described herein. The pharmaceutical composition comprises cannabinoids and/or terpenes.

Various aspects of the disclosure relate to a method of producing oil. The method comprises crushing seed or plant material as defined herein to release the oil.

Various aspects of the disclosure relate to a method of producing oil. The method comprises extracting oil from a plant or plant cell as described herein.

Various aspects of the disclosure relate to a method of producing an extract comprising a cannabinoid and/or a terpene. The method comprises extracting the cannabinoid and/or the terpene from seed or plant material as described herein.

Various aspects of the disclosure relate to a method of producing an extract comprising a cannabinoid and/or a terpene. The method comprising extracting the cannabinoid and/or the terpene from a plant or plant cell, or dried flower, as described herein.

Various aspects of the disclosure relate to a method of producing a concentrate comprising a cannabinoid and/or a terpene, The method comprises extracting a concentrate comprising the cannabinoid and/or the terpene from seed or plant material as described herein.

Various aspects of the disclosure relate to a method of producing a concentrate comprising a cannabinoid and/or a terpene. The method comprises extracting a concentrate comprising the cannabinoid and/or the terpene from a plant or plant cell, or dried flower, as described herein.

Various aspects of the disclosure relate to a method of producing an isolate comprising a cannabinoid and/or a terpene. The method comprises isolating the cannabinoid and/or the terpene from seed or plant material as described herein.

Various aspects of the disclosure relate to a method of producing an isolate comprising a cannabinoid and/or a terpene. The method comprises isolating the cannabinoid and/or the terpene from a plant or plant cell, or dried flower, as described herein.

Other aspects and features of the present invention will become apparent to those ordinarily skilled in the art upon review of the following description of specific embodiments of the invention

### BRIEF DESCRIPTION OF THE TABLES

Table 1 provides example genomic-based context sequences (SEQ ID NOS:5-14) that contain polymorphic sites within a 20 megabase region within the chromosome identified by GenBank Sequence as CM010796.2 and located between 40,320,373 bases to 59,999,986 bases, and extensive polymorphism information that includes observed alleles and information about the type of polymorphism. The precise positions of the polymorphisms are indicated in bold and underlined type.

Table 2 provides example genomic-based context sequences (SEQ ID NOS:15-24) that contain polymorphic sites within approximately 10 kbp upstream and downstream of the Cannabis UPF2 gene, and extensive polymorphism information that includes observed alleles and information about the type of polymorphism. The precise positions of the polymorphisms are indicated in bold and underlined type.

Table 3 provides example genomic-based context sequences (SEQ ID NOS:25-34) that contain polymorphic sites associated with the day length neutral (“autoflowering”) phenotype located within approximately 500 kbp upstream and 500 kbp downstream of the Cannabis UPF2 gene, and extensive polymorphism information that includes observed alleles and information about the type of polymorphism. The precise positions of the polymorphisms are indicated in bold and underlined type.

Table 4 provides example genomic-based context sequences (SEQ ID NOS:35-44) that contain polymorphic sites associated with the day length neutral (“autoflowering”) phenotype located on the chromosome identified by GenBank Sequence as CM010796.2 of the Cannabis genome, and polymorphism information that includes observed alleles and information about the type of polymorphism. The precise positions of the polymorphisms are indicated in bold and underlined type.

### DEFINITIONS

“Cannabis” as used herein is inclusive of all species and varieties falling within the genus Cannabis, whether Cannabis sativa, Cannabis indica, or Cannabis ruderalis; C. ruderalis, and whether or not the variety is “drug” (i.e. contains appreciable levels of the psychoactive cannabinoid tetrahydrocannabinol (“THC”)) or “non-drug” (e.g. “hemp”).

“Photoperiod” as used herein refers to the day length to which a plant is exposed. Many flowering plants, including Cannabis, sense seasonal changes in day length, i.e. photoperiod, which they may take as a signal to flower. For example, most Cannabis varieties in cultivation initiate flowering upon a transition from long days (long day length) to short days (short day length). The skilled person understands that the specific length of the “long days” or the “short days” is not crucial. Rather it is the change in the day length that is important to the initiation of flowering.

An “autoflowering phenotype” or “day length neutral phenotype” as used herein describes Cannabis plants that initiate flowering with developmental age regardless of the photoperiod (or change therein) to which the plants are exposed.

A “photoperiod phenotype” or “day length sensitive phenotype” as used herein describes Cannabis plants that initiate flowering with the onset of decreasing day length (i.e. reduced photoperiod).

“Long days” as used herein refers to day lengths of between about 16 h and about 18 h (i.e. between 6 h and 8 h of darkness).

“Short days” as used herein refers to day lengths of about 12 h (i.e. about 12 of darkness).

“Genetic material” as used herein includes any nucleic acid, but particularly deoxyribonucleic acid (DNA) in double-stranded form.

A “polymorphic site” or “polymorphism” as used herein refers to a position within a given genomic region at which variation exists within a population. A polymorphic site” or “polymorphism” is the occurrence of two or more forms (alleles) at a position in the genome within a population, in such frequencies that the presence of the rarest of the forms cannot be explained by mutation alone. Polymorphic sites have at least two alleles, each occurring at a frequency of greater than 1%. Polymorphic sites may occur in both coding regions and noncoding regions of genes or in intergenic regions. Polymorphic sites may involve a single nucleotide polymorphism (i.e. a SNP), or may involve an insertion or deletion (“indel”), or rearrangement.

A “single nucleotide polymorphism” (“SNP”) as used herein refers to a polymorphism that involves a variation at a single nucleotide position, whether it be an insertion, a deletion, or a substitution.

A “causative polymorphism” as used herein refers to a variation at a polymorphic site that produces an alteration in the expression of a gene product (whether at the transcriptional, translational, or protein product level) to result in or contribute to a relevant phenotype. Thus, a causative polymorphism is the most predictive of a phenotype.

“Causative” as used herein means resulting in or contributing to a relevant phenotype.

“Linkage” as used herein refers to the co-inheritance of alleles at two or more genes or sequences due to the proximity of the genes on the same chromosome.

“Linkage disequilibrium” (“LD”) as used herein refers to any deviation from the expected frequency, i.e. if they were segregating completely independently, of the alleles of two genes in a population. LD is defined in the context of the relative frequency of gamete types in a population of many individuals in a single generation, and is discussed extensively, for example, in WO2009/123396. Loci that have a high degree of LD with an allele of interest are potentially useful in predicting the presence of the allele of interest (i.e. associated with the condition or side effect of interest). Accordingly, two polymorphisms that have a high degree of LD may be equally useful in determining the identity of the allele of interest. Accordingly, the determination of the allele at a polymorphic site can provide the identity of the allele at any polymorphic site in LD therewith. The higher the degree of LD, the more likely that two polymorphisms may reliably function as surrogates for each other. LD may be useful for genotype-phenotype association studies. Until the mechanism underlying the genetic contribution of the day length neutral phenotype is fully understood, LD can be helpful in identifying potential candidate causative variations and in identifying a range of polymorphisms that may be commercially useful for predicting the day length neutral phenotype of a variety.

“Haplotype” as used herein refers to a set of alleles of closely linked loci on a chromosome that tend to be inherited together. Haplotypes along a given segment of a chromosome are generally transmitted to progeny together unless there has been a recombination event. In the absence of a recombination event, haplotypes can be treated as alleles at a single highly polymorphic site for mapping.

The term “traditional breeding techniques” as used herein includes crossing, selfing, selection, backcrossing, marker assisted breeding/selection, mutation breeding etc. as known to the breeder (i.e. methods other than genetic modification/transformation/transgenic methods), by which, for example, a genetically heritable trait can be transferred from one Cannabis line or variety to another.

“Backcrossing” as used herein is a traditional breeding technique used to introduce a trait into a plant line or variety. A first parent containing a trait of interest is crossed to a second parent to produce progeny plants. Progeny plants which have the trait are then “backcrossed” to one or the other parent, usually the second parent. After several generations of backcrossing and/or selfing, the progeny typically have the genotype of the second parent but with the trait of interest from the first parent.

“Concentrate” as used herein refer to a product derived from Cannabis flowers, whereby excess plant material and other impurities have been removed to leave primarily the cannabinoids and/or terpenes.

“Plant material” as used herein refers to any portion or combination of portions of a Cannabis plant including but not limited to seeds, stems, stalks, leaves, flowers, roots, whether fresh or dry, or whether intact, cut, or comminuted. A “field” or a “crop” of plants as used herein refers to a plurality of Cannabis plants cultivated together in close proximity.

An “endogenous” gene as referred to herein refers to a gene that is naturally present in a population of Cannabis plants and has not been introduced through genetic modification. An “endogenous” gene may be distinguished from a second copy of the gene that is introduced by, for example, genetic modification, and exists at a separate locus in the genome. For the purposes of this disclosure, unless context dictates otherwise, the terms “endogenous UPF2 gene”, “Cannabis UPF2 gene”, and “UPF2 gene” are used interchangeably and the terms “endogenous gene” and “Cannabis gene” are used interchangeably

A “genetic modification” as used herein broadly refers to any novel combination of genetic material obtained with techniques of modern biotechnology. Genetic modifications include, but are not limited to, “transgenes” in which the genetic material has been altered by the insertion of exogenous genetic material. However, genetic modifications also include alterations (e.g. insertions, deletions, or substitutions) in endogenous genes introduced in a targeted manner with techniques such as CRISPR/Cas9, TALENS, etc. as discussed elsewhere herein. However, for the purposes of this disclosure “genetic modification” is not intended to include novel combinations of genetic material resulting from mutations generated by traditional means of random mutagenesis following by traditional means of breeding. Genetic modifications may be transient or stably inherited.

A “genetically modified” plant or plant cell as used herein broadly refers to any plant or plant cell that possesses a genetic modification as defined herein.

“Nucleotide sequence”, “polynucleotide sequence”, “nucleic acid” or “nucleic acid molecule” as used herein refers to a DNA or RNA polymer which can be single or double stranded and optionally contains synthetic, non-natural or altered nucleotide bases capable of incorporation into the DNA or RNA polymer.

A “fragment”, a “fragment thereof”, “gene fragment” or a “gene fragment thereof” as used herein refers to a portion of a “nucleotide sequence”, “polynucleotide sequence”, “nucleic acid” or “nucleic acid molecule”. For example, a “fragment” as used herein with respect to the UPF2 gene sequence refers to a portion of the UPF2 gene sequence that is less than the complete sequence. In various embodiments, the fragment may be useful to reduce expression of the a gene of interest, e.g. the UPF2 gene. In various embodiments, the fragment comprises at least 20, at least 40, at least 60, at least 80, at least 100, at least 150, at least 200, at least 250, at least 300, at least 350, at least 400, at least 450 or at least 500 contiguous nucleotides.

“Allele” or “variant” as used herein refers to a nucleotide sequence at a polymorphic site where at least two nucleotide sequences exist at the polymorphic site in a population at appreciable frequencies.

A “non-natural variant” as used herein refers to nucleic acid sequences native to an organism but comprising modifications to one or more of its nucleotides introduced by mutagenesis (including point mutations, insertions, and deletions).

“Identity” as used herein refers to sequence similarity between two polynucleotide molecules. Identity can be determined by comparing each position in the aligned sequences. A degree of identity between nucleic acid sequences is a function of the number of identical or matching amino acids or nucleic acids at positions shared by the sequences over a specified region.

“Heterologous” or “exogenous” as used herein refers to DNA that does not occur naturally as part of the plant's genome, or is not normally found in the host genome in an identical context.

“Transgene” as used herein refers to a segment of DNA containing a gene sequence that has been isolated from one organism and introduced into a different organism. In the context of the present disclosure, the nucleic acid molecules may comprise nucleic acid that is heterologous to the plant in which gene expression (for example, without limitation, UPF2 gene expression) is reduced.

“Expression” or “expressing” as used herein refers to the process by which information from a gene is used in the synthesis of a functional gene product, and may relate to production of any detectable level of a product, or activity of a product, encoded by a gene. Gene expression may be modulated (i.e. initiated, increased, decreased, terminated, maintained or precluded) at many levels including transcription, RNA processing, translation, post-translational modification, protein degradation. In the context of the present disclosure, reduced expression of an endogenous Cannabis gene (for example, without limitation, the endogenous UPF2 gene) can be effected by reduced transcription of the endogenous Cannabis gene (for example, without limitation, the endogenous UPF2 gene), by reduced translation of mRNA transcripts (for example, without limitation, UPF2 mRNA transcripts), or by the introduction of mutations that either prevent the translation of functional polypeptides or result in the translation of polypeptides with reduced abilities to convert substrate. Such reduced expression of a endogenous Cannabis gene (for example, without limitation, the endogenous UPF2 gene) may result from expression of transgenes comprising expression constructs designed to reduce expression of the endogenous genes.

“Decreasing expression”, “decreasing activity”, “reducing expression”, and “reducing activity” as used herein are intended to encompass well known equivalent terms regarding expression and activity such as “inhibiting”, “down-regulating”, “knocking out”, “silencing”, etc.

“Expression construct” as used herein refers to any type of genetic construct containing a nucleic acid coding for a gene product in which part or all the nucleic acid encoding sequence is capable of being transcribed. The transcript may be translated into a protein, but it need not be. An expression construct of the disclosure nucleic acid molecule may further comprise a promoter and other regulatory elements, for example, an enhancer, a silencer, a polyadenylation site, a transcription terminator, a selectable marker or a screenable marker.

“Promoter” as used herein refers to a nucleotide sequence that directs the initiation and rate of transcription of a coding sequence. The skilled person will understand that it would be important to use a promoter that effectively directs the expression of the construct in the tissue in which a Cannabis gene (for example, without limitation, the UPF2 gene) is usually expressed. For example, the endogenous UPF2 gene promoters could be used. Alternatively, constitutive, tissue-specific, or inducible promoters useful under the appropriate conditions to direct high-level expression of the introduced expression construct could be used.

“Constitutive promoter” as used herein refers to a promoter which drives the expression of the downstream-located coding region in all or nearly all tissues regardless of environmental or developmental factors.

### DETAILED DESCRIPTION

### Initiation of Flowering in Response to Day Length

Many Cannabis varieties in production today have a day length sensitive phenotype. Photoperiod sensitivity is especially limiting for crops grown outdoors at more northern and southern latitudes. Near the equator, the variation of temperature and light (e.g. 12 hours of sun and 12 hours of darkness per day) is minimal, such that the plants do not experience a change in the photoperiod, but they rather flower when the plant reaches maturity. However, at more northerly and southerly latitudes, Cannabis is effectively a seasonal plant that needs favourable temperature conditions to grow, and light conditions to flower. Since flowering can be triggered by a change (i.e. a decrease) in day length (i.e. photoperiod), growing conditions when flowering is triggered may be less than optimal for harvest. For example, a sufficient decrease in day length to initiate flowering may not occur until late summer or early fall when frost may occur impacting a plant's quality and/or yield.

In contrast, day length neutral (i.e. “autoflowering”) plants can be used at more extreme latitudes since they don't depend on photoperiod to flower. Such plants flower automatically with age, typically have short lifecycles, and, therefore, may mature before the end of the outdoor season yielding a better harvest index. In other words, autoflowering plants can be planted and harvested in a manner that takes advantage of the best growing conditions at such extreme latitudes.

Methods for selecting for day length neutral (“autoflowering”) plants are thus needed to develop varieties for more northern/southern latitudes so that plants can be cultivated outdoors and thereby avoid the costs and resources required by indoor cultivation (e.g. in terms of energy consumption, space, and labor that is required for growth in a greenhouse). The suitability of a variety for cultivation in a wide variety of conditions may contribute to high productivity.

Day length neutral phenotypes are also valuable for growth in greenhouses at more extreme latitudes. Shade curtains are required in greenhouses to control the amount of light to which a crop is exposed each day in order to avoid initiation of flowering prematurely, or to simulate a decrease in day length if the initiation of flowering is desired. The cultivation of plants with day length neutral phenotypes avoids the need for such shade curtains.

Thus, breeding Cannabis varieties with day length neutral (“autoflowering”) phenotypes has significant economic value. Plants with polymorphisms that are predictive of the day length neutral phenotype can be genotyped early in the life of the plant, thereby allowing for the selection of plants desired for further breeding long before the onset for flowering, and for the culling of photoperiod phenotype plants that do not carry the autoflowering trait.

Thus, there is a need for novel genetic markers that are predictive of the day length neutral phenotype. The identification of such genetic markers associated with autoflowering may also reveal novel mutations that are causative of the day length neutral phenotype, and thus identify novel gene targets for the manipulation of response to photoperiod.

### Polymorphisms

The genomes of all organisms undergo spontaneous mutations in the course of their evolution to generate variant forms of progenitor genetic sequences, whether they be substitutions of one nucleotide for another at the polymorphic site, or insertions or deletions of as few as one nucleotide. In many cases, both progenitor and variant forms survive and co-exist in a species population. The coexistence of multiple forms of a genetic sequence segregating at appreciable frequencies is defined as a “genetic polymorphism” at a “polymorphic site”, which includes single nucleotide polymorphisms (“SNPs”), i.e. single base positions in DNA at which different alleles, or alternative nucleotides, exist in a population.

Polymorphic sites may have several alleles. However, the vast majority of polymorphic sites are bi-allelic.

Regardless of whether the polymorphism involves a SNP, an insertion, or a deletion, the polymorphic site is usually preceded and followed by highly conserved “context” sequences (e.g., sequences that vary in less than 1/100 or 1/1000 members of the populations). An individual may be homozygous or heterozygous for an allele at each polymorphic site.

Causative polymorphisms may typically be positioned within genes encoding a polypeptide product, for example, SNPs that result in changes in the amino acid sequence that result in a defective product. However, causative polymorphisms do not necessarily have to occur in coding regions, and can occur in any region that affects the expression of the protein encoded by a gene, whether by transcription or translation.

Some polymorphisms that are not causative polymorphisms nevertheless are in LD with, and therefore segregate with, the phenotype of concern. Thus, such polymorphisms may be commercially useful for predicting the phenotype.

An association study of a polymorphism and a specific phenotype involves determining the presence or frequency of the polymorphic allele in biological samples from individual plants with the phenotype of interest, such as a day length neutral phenotype, and comparing the information to that of controls (e.g. individual plants that have a day length sensitive phenotype).

A polymorphic site may be surveyed in a sample obtained from an individual having a day length neutral phenotype, compared to control (i.e. day length sensitive plant) samples, and selected for its increased (or decreased) prevalence. Once a statistically significant association is established between one or more polymorphisms and the phenotype of interest, e.g. day length neutral, then the genomic region around the polymorphic site(s) can be thoroughly screened to identify the causative polymorphisms or additional polymorphic sites in LD with the identified polymorphic sites.

### Polymorphism Analysis

Once an individual plant is identified as a candidate for having an day length neutral phenotype, or being a carrier of a day length neutral allele, then genetic sequence information may be obtained from the plant for the purpose of determining the identity of the alleles at one or more polymorphic sites associated with the day length neutral phenotype. Genetic sequence information may be obtained in numerous different ways and may involve the collection of a biological sample that contains genetic material, particularly, genomic DNA containing the sequence or sequences of interest. Any suitable genomic DNA samples from Cannabis may be used, e.g. from seeds, seedlings, tissue cultures or plants of any age, preferably before flowering has been initiated. Many methods are known in the art for collecting biological samples and extracting genetic material from those samples, as surveyed, for example, in WO 2009/124396 and WO2016/197258. As discussed in WO2019/222835, samples may include the leaves of Cannabis seedlings or young plants pressed into a paper matrix, such as a paper matrix (e.g. Whatman™ FTA™ cards) comprising a mixture of chemicals that lyse cells and stabilize nucleic acids on contact for long-term storage at room temperature. The ability to use leaf material pressed into a paper matrix is particularly advantageous because the samples are easily collected, stored at room temperature, and shipped in a format that is not subject to the same restrictions as handling of controlled or regulated substances. Alternatively, the samples may comprise purified DNA isolated from fresh or dry Cannabis plant material.

Once an individual plant's genomic DNA has been obtained, the identity of the allele at one or more of the polymorphic sites associated with the day length neutral phenotype can be determined using any one of several methods known in the art. A variety of methods for determining the sequence at polymorphic sites are discussed in, for example, WO 2009/124396, WO2011/133418, WO2016/197258, and WO2019/222835.

Detection or determination of a nucleotide identity, or the presence of one or more SNP(s), may be accomplished by any one of a number of methods or assays known in the art. Many methodologies are useful for determining the identify of alleles at polymorphic sites, and can be assigned to one of four broad groups (sequence-specific hybridization, primer extension, oligonucleotide ligation and invasive cleavage). Furthermore, there are numerous methods for detecting the products of each type of reaction (e.g. fluorescence, luminescence, etc.).

In one popular method discussed in WO2019/222835, High Resolution Melt (HRM) analysis may be conducted in the presence of a common intercalating fluorescent dye to determine the identity of the alleles present at a polymorphic site. As double stranded DNA denatures during HRM, the loss in fluorescence from the dye over time provides different denaturation curves depending on which alleles are present at the polymorphic site.

In another favored method, 5′ exonuclease activity or TaqMan™ assay (Applied Biosystems) displaces and cleaves the oligonucleotide probes hybridized to a specific target DNA generating to fluorescent signal. The skilled person understands that numerous methods for determining the identity of an allele at a polymorphic site are known in the art. The skilled person understands that the particular method of determining the identity of the allele at the polymorphic site is not important, so long as it may be reliably determined.

### Marker Assisted Breeding

Breeding new varieties, lines and hybrids may be achieved using techniques of crossing and selection on a set of parental lines taking advantage of the plant's method of pollination (self-, sib- or cross-pollination). Multiple rounds of crossing and selection may be needed to produce plant varieties with the desired traits. After each round, the breeder selects candidates with the desired traits or markers for the next round of selection.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the allele of the desired trait (e.g. day length neutral phenotype) and against the alleles of the undesired trait (e.g. day length sensitive phenotype). The use of this procedure can reduce the number of back crosses to the desired parent in a backcrossing program.

The skilled person understands that other desired traits (e.g. altered height, early or late maturity, or disease resistance) can be transferred into selected autoflowering lines by cross pollinating plants having a day length neutral (autoflowering) phenotype with a second parent plant having the desired traits, collecting F₁ seed, growing a F₁ plant which is allowed to self-pollinate and collect the F₂ seed. The F₂ seed would then be grown, and individual plants that have the day length neutral phenotype could be selected according to the methods herein. The skilled person will be able to backcross the selected individuals to the second parent.

Identification of Polymorphic Sites in the Cannabis Genome that are Associated with the Autoflowering (“Day Length Neutral”) Phenotype

A plurality of F₁ populations featuring one parent with a day length neutral (i.e. autoflowering) phenotype and a second parent with a day length sensitive (i.e. photoperiod) phenotype were generated. Photoperiod sensitive plants were treated with silver thiosulfate (“STS”), a suppressor of ethylene production by plants, to induce the formation of male flowers. Pollen from the male flowers of the photoperiod sensitive plants was then used to pollinate five female plants having a day length neutral phenotype.

The resulting populations were grown under long days (photoperiod=16 h to 18 h). Under these long days, all of the F₁ individuals remained vegetative, indicating that they were all photoperiod sensitive, and that the autoflowering trait is recessive. The photoperiod was then switched to short days (photoperiod=12 h), under which the F₁ individuals initiated flowering. The F₁ individuals were treated with STS to induce the production of male flowers, and placed in pollination bags to self-pollinate to produce F₂ seed.

Three F₂ populations were grown indoors under long days (photoperiod=16 h to 18 h). Three F₂ populations were grown outdoors and scored for flowering when day length was approximately 14 h. In each F₂ population, 25% of the plants flowered under the long days. That is, the F₂ populations segregated 3:1 photoperiod phenotype: day length neutral phenotype (170 individuals being photoperiod sensitive and 61 day length neutral), which indicates that the day length neutral phenotype is a recessive trait and controlled by a single locus.

A Genome-Wide Association Study (GWAS) was performed using 18,106 “genotyping by sequencing” (“GBS”) variants in a Mixed Linear Model in TASSEL5 with 278 samples (47 autoflowering, 231 photoperiod). The kinship (K) matrix was generated using centered Identity-By-State (IBS). The first 5 principal components of the genotype data were included in the model to control population structure. For mapping analysis, phenotypes were coded as 1 for day length neutral phenotype samples and 0 for photoperiod phenotype samples. A number of SNPs in the chromosome identified by GenBank Sequence as CM010796.2 (“chromosome CM010796.2”) were identified by GWAS analysis as significantly associated with the day length neutral phenotype. Additionally, an association analysis using a General Linear Model was performed using 302,334 whole-genome shotgun variants on chromosome CM010796.2 in TASSEL5 with 39 samples (12 autoflowering, 27 photoperiod). For mapping analysis phenotypes were coded as 1 for day length neutral phenotype samples and 0 for photoperiod phenotype samples. 1,867 variants were found to be significantly correlated with the day length neutral phenotype in a region spanning approximately position 40,320,373 to 59,999980.

To develop a genetic marker to select plants containing the day length neutral phenotype, a genetic map of the region associated with the autoflowering trait in chromosome CM010796.2 was constructed. The genetic map of this region informed the size of the region carrying the autoflowering trait, its recombination rate and the genetic distance between markers. This information was used to select suitable SNP(s) for use in marker assisted selection and to establish a breeding protocol. To construct the genetic map of chromosome CM010796.2, 192 seeds from an F₂ population obtained from a single F₁ selfed individual from an initial cross between a photoperiod sensitive parent and a day length neutral parent were germinated. The seedlings were genotyped using a custom-made 5KSeqSNP array from LGC™ to assess the recombination rate of chromosome CM010796.2 and to measure the genetic distance between the SNPs identified by the GWAS analysis. The 5KSeqSNP array included some of the polymorphic SNPs in day length neutral plants.

Segregating alleles in the F₂ population were used to build a genetic map of chromosome CM010796.2 using a Kosambi map function as implemented in a R-qtl package. SNPs with genotypic data in at least 160 individuals were retained for map construction. A total of 90 SNPs were segregating in chromosome CM010796.2 and allowed the construction of a genetic map with an average genetic distance between markers of 1 centimorgan (cM). The recombination rates were analyzed across chromosome CM010796.2. Two major regions of low recombination were observed. This indicates that, in addition to the centromere's “natural’ low recombination zone, there is a distinct region comprising about 20 megabases in one of the chromosome arms of chromosome CM010796.2 that displays very low recombination rates. 24 of the 90 SNPs with very little to no recombination fell within this about 20 megabase region. This about 20 megabase region is located between about 40 megabases to about 60 megabases in chromosome CM010796.2. In some embodiments, this about 20 megabase region is located between about 40,320,373 bases to about 59,999,986 bases. Without being bound by theory, the inventors speculate that the second low recombination region (i.e. the about 20 megabase region in chromosome CM010796.2) may be attributable to the autoflowering trait originating from an ancestral Cannabis line, presumably from Cannabis ruderalis, which would be divergent enough to disrupt the normal process of cross-over and recombination. Thus, the autoflowering trait (i.e. the day length neutral phenotype) is attributed to the low recombination region comprising about 20 megabases (i.e. the “about 20 megabase region”).

In some embodiments, the low recombination region in chromosome CM010796.2 that may be attributable to the autoflowering trait is about 1 megabase, about 2.5 megabases, about 5 megabases, about 7.5 megabases, about 10 megabases, about 12.5 megabases, about 15 megabases, about 17.5 megabases or about 20 megabases located between about 40 megabases to about 60 megabases. In some embodiments, the low recombination region is located within a region in Cannabis chromosome CM010796.2 selected from: about 40 megabases to about 42.5 megabases, about 40 megabases to about 45 megabases, about 40 megabases to about 50 megabases, about 42.5 megabases to about 45 megabases, about 42.5 megabases to about 47.5 megabases, about 42.5 megabases to about 50 megabases, about 42.5 megabases to about 52.5 megabases, about 45 megabases to about 47.5 megabases, about 45 megabases to about 50 megabases, about 45 megabases to about 52.5 megabases, about 45 megabases to about 55 megabases, about 47.5 megabases to about 50 megabases, about 47.5 megabases to about 52.5 megabases, about 47.5 megabases to about 55 megabases, about 47.5 megabases to about 57.5 megabases, about 50 megabases to about 52.5 megabases, about 50 megabases to about 55 megabases, about 50 megabases to about 57.5 megabases, about 50 megabases to about 60 megabases, about 52.5 megabases to about 55 megabases, about 52.5 megabases to about 57.5 megabases, about 52.5 megabases to about 60 megabases, about 55 megabases to about 57.5 megabases, about 55 megabases to about 60 megabases, or about 57.5 megabases and about 60 megabases.

Due to the low recombination of the about 20 megabase region in chromosome CM010796.2, one or more polymorphism in that region (or in any one or more of the 1,172 genes within this region) in the Cannabis genome may be causative of and/or contribute to the day length neutral phenotype. Thus, any polymorphism in this low recombination region would be in strong linkage disequilibrium with the causative gene(s) and could be used as a marker to select individuals with the day length neutral phenotype in a segregating population.

For example, about 1,867 variants have been identified in the about 20 megabase region in chromosome CM010796.2. Any one or more of these variants may be causative of the day length neutral phenotype and/or in strong linkage disequilibrium with the causative gene(s) and could be used as a marker to select individuals with the day length neutral phenotype.

Example polymorphic sites associated with the day length neutral phenotype, and within the about 20 megabase region (i.e. between about 40 megabases and about 60 megabases in chromosome CM010796.2), and the alleles present at these sites, are presented in Table 1. The skilled person understands that additional polymorphic sites not identified in Table 1 but that are within the about 20 megabase region, and the alleles present at these sites, may be used for the identification of plants that have the day length neutral phenotype or that are carriers of the trait for day length neutral phenotype.

A putative SNP in the Cannabis UPF2 gene was found across different genome assemblies (Laverty and DT2 improved) from “genotyping by sequencing” (“GBS”) data. To validate this putative SNP, the following primers around the putative SNP were designed: SW23 (CTAAAAAAAGTAGCGCTTCGGG; SEQ ID NO: 45) and SW28 (TGTAGCCGTGGGGCAACTAAAACG; SEQ ID NO: 46). PCR amplification of selected F₂ plants (7 autoflowering, 9 photoperiod) was performed using the primers. 1 ul DNA was amplified with 1 uM of each primer, 0.2 mM dNTP, 3 mM MgCl₂ and 2 Units Taq polymerase, using the following cycling program: 5 min at 98° C., then 30 cycles of 10 sat 98° C., 30 s at 58° C., 1 min at 72° C. and final extension 10 min at 72° C. Surprisingly, the expected PCR product was absent in all plants having the day length neutral phenotype.

To test if the absence of the PCR product in plants with a day length neutral phenotype was caused by mutations at the primer region, a new set of primers flanking the previous sequences were designed as SW36 (GTACAGTAAACTATCTCAATTTCT; SEQ ID NO: 3) and SW37 (ACCACACCTTTTCCAATTGGACTC; SEQ ID NO: 4). By employing the same method as above, PCR products were detected using the new pair of primers in autoflowering samples. The SW28 primer sequence was absent in the PCR products, which indicates that plants having the day length neutral phenotype have a deletion at this polymorphic site. More samples (i.e. 8 autoflowering, 11 photoperiod) were tested, and validated the result.

Upon further investigation, this deletion was determined to be located in the 3′ UTR of the Cannabis UPF2 gene and comprise the sequence of SEQ ID NO: 2 (i.e. SEQ ID NO: 2 is missing from both copies of the UPF2 gene in plants displaying a day length neutral phenotype). While not conclusive, the data indicates that the deletion of this DNA fragment is an important determinant, and may be causative, of the day length neutral phenotype.

The Cannabis UPF2 gene encodes a nonsense-mediated mRNA decay (NMD) factor protein. NMD is an important mRNA quality surveillance pathway in all eukaryotes that eliminates aberrant mRNAs. In Arabidopsis, silencing the UPF2 gene results in aberrant stress responses under long (16 h) days, but has a lesser effect under short days. As such, it appears that UPF2 may play a role in mediating the response to long days (i.e. 16 h) in Arabidopsis. Accordingly, it is reasonable to expect that UPF2 may play a role in mediating inhibition of flowering in response to long days (16 to 18 h) in Cannabis.

However, UPF2 is within an about 20 megabase region containing 1,172 genes and numerous mutations, any of which could be the causative allele responsible for the day length neutral phenotype. For example, UPF2 is within an about 1 megabase region (located between about 41,177,432 bases and about 42,183,459 bases in chromosome CM010796.2) containing 46 additional genes and numerous mutations, any of which could be the causative allele responsible for the day length neutral phenotype.

Moreover, once the allele of the Cannabis gene within the about 20 megabase region is established as associated with the day length neutral phenotype, the skilled person understands that any allele at additional polymorphic sites on the chromosome that is in linkage disequilibrium with this allele will also be associated with the day length neutral phenotype. For example, once the allele of the UPF2 gene from which SEQ ID NO: 2 is deleted is established as associated with the day length neutral phenotype, the skilled person understands that any allele at additional polymorphic sites on the chromosome that is in linkage disequilibrium with this allele of UPF2 will also be associated with the day length neutral phenotype.

Accordingly, GWAS and GBS allowed for the identification of more polymorphic sites that are in linkage disequilibrium with the deletion of SEQ ID NO: 2 from the UPF2 gene. Example polymorphic sites within 10 kbp upstream and downstream of the UPF2 gene, and the alleles present at these sites, are presented in Table 2. Additional example polymorphic sites associated with the day length neutral phenotype and within 500 kbp upstream and 500 kbp downstream of the UPF2 gene, and the alleles present at these sites, are presented in Table 3. Further example polymorphic sites on chromosome CM010796.2 that are significantly associated with the day length neutral phenotype are presented in Table 4. The skilled person understands that it may be possible to use any one of these polymorphisms as a surrogate marker for the deletion in the UPF2 gene for the identification of plants that have the day length neutral phenotype or that are carriers of the trait for the day length neutral phenotype. The skilled person also understands that additional polymorphic sites not identified in any of Tables 2, 3 or 4 but that are within 3,000 bp upstream and 3,000 bp downstream of the UPF2 gene, and the alleles present at these sites, may be used for the identification of plants that have the day length neutral phenotype or that are carriers of the trait for day length neutral phenotype.

Identification of Plants that have an Autoflowering (“Day Length Neutral”) Phenotype

Thus, the skilled person understands that the polymorphisms disclosed in Tables 1, 2, 3, and 4 are useful in methods of identifying whether or not a Cannabis plant has a day length neutral phenotype, or is a carrier of a trait for a day length neutral phenotype, but that other polymorphisms within the about 20 megabase region may be useful in such methods.

In one aspect, this disclosure provides a method comprising testing nucleic acid from a Cannabis plant to determine the presence or absence a polymorphic site in an about 20 megabase region (or in any one or more of the 1,172 endogenous genes in that region) in chromosome CM010796.2 located between about 40 megabases and about 60 megabases. For example, the disclosure provides a method comprising testing nucleic acid from a Cannabis plant to determine the presence or absence of a deletion in the 3′ untranslated region of the endogenous UPF2 gene comprising a portion (i.e. all or a part) of SEQ ID NO: 2. In particular, the methods are for identifying whether or not the Cannabis plant has a day length neutral phenotype, or is a carrier of a trait for a day length neutral phenotype. The presence of a variation indicates that the plant has a day length neutral phenotype or is a carrier of the trait for the day length neutral phenotype. In some embodiments, testing nucleic acid from the Cannabis plant to determine the presence or absence of the variation comprises testing for the presence or absence of SEQ ID NO: 2 from SEQ ID NO: 15, or a portion thereof wherein the absence of SEQ ID NO: 2 or a portion thereof from SEQ ID NO: 15 in both copies of the UPF2 gene indicates that the plant has a day length neutral phenotype. The absence of SEQ ID NO: 2 or a portion thereof from SEQ ID NO: 15 in only one copy of the UPF2 gene indicates that the plant is a carrier of the trait for a day length neutral phenotype.

However, the skilled person understands that if the UPF2 allele comprising the deletion of SEQ ID NO: 2 is causative of the day length neutral phenotype, i.e. if the day length neutral phenotype results from a loss or gain of UPF2 gene expression/activity, then any loss or gain of function of the UPF2 gene could function as a marker of the day length neutral phenotype. If the presence or absence of an allele at a polymorphic site within the about 20 megabase region (or within any one or more of the 1,172 endogenous genes in the region) is causative of the day length neutral phenotype, i.e. if the day length neutral phenotype results from a loss or gain of any one or more of the 1,172 endogenous gene expression/activity, then any loss or gain of function of such gene could function as a marker of the day length neutral phenotype. The skilled person would also recognize that any SNPs in close proximity to a causative gene (i.e. within about 3,000 bases of the 5′ and 3′ ends of the gene and/or within the promoter and/or untranslated region flanking the gene) could also function as a marker of the day length neutral phenotype. Thus, in another aspect, this disclosure more generally provides a method comprising testing nucleic acid from a Cannabis plant to determine the presence or absence of an allele at a polymorphic site in the about 20 megabase region (or in any one or more of the 1,172 endogenous genes within the region). For example, another aspect of the disclosure generally provides a method comprising testing nucleic acid from a Cannabis plant to determine the presence or absence of a variation in the endogenous UPF2 gene. The methods are for identifying whether or not the Cannabis plant has a day length neutral phenotype, or is a carrier of a trait for a day length neutral phenotype. The presence of the allele at a polymorphic site or variation indicates that the plant has a day length neutral phenotype or is a carrier of the trait for the day length neutral phenotype. The variation may be a loss of function mutation or a gain of function mutation in any one or more of the 1,172 endogenous genes in the about 20 megabase region (for example, without limitation, a loss of function mutation or a gain of function mutation in the UPF2 gene), which may be a point mutation (e.g. a substitution, a missense mutation or a nonsense mutation), a deletion, or an insertion. The presence of the variation in both copies of the endogenous gene (for example, without limitation, in both copies of the endogenous UPF2 gene) may indicate that the plant has a day length neutral phenotype. The presence of the variation in only a single copy of the endogenous gene (for example, without limitation, the endogenous UPF2 gene) may indicate that the plant is a carrier of the trait for the day length neutral phenotype.

The skilled person further understands that any polymorphism in linkage disequilibrium with an allele at a polymorphic site of the about 20 megabase region can be used as a surrogate marker for the day length neutral phenotype. For example, any polymorphism in linkage disequilibrium with the allele of the UPF2 gene with the deletion in the 3′ untranslated region (i.e. the deletion of SEQ ID NO: 2) can be used as a surrogate marker for the day length neutral phenotype. Thus, in another aspect, this disclosure provides a method comprising testing nucleic acid from a Cannabis plant to determine the presence or absence of one or more variation (i.e. allele at a polymorphic site) that is in linkage disequilibrium (i.e. where the coefficient of correlation of the alleles, r², is 0.9 to 1) with a variation in the about 20 megabase region (for example, without limitation, with the UPF2 allele comprising the deletion of SEQ ID NO: 2). The method is for identifying whether a Cannabis plant has a day length neutral phenotype, or is a carrier of a trait for a day length neutral phenotype. The presence of the variation indicates that the plant has a day length neutral phenotype or is a carrier of the trait for the day length neutral phenotype. The presence of the allele indicates that the plant has a day length neutral phenotype or is a carrier of the trait for the day length neutral phenotype.

Again, the skilled person understands that if this deletion of SEQ ID NO: 2 is causative of the day length neutral phenotype, then the presence or absence of one or more variation (i.e. allele at a polymorphic site) that is in linkage disequilibrium (i.e. r²=0.9 to 1) with any loss or gain of function variation in the endogenous UPF2 gene could be used as a marker for the day length neutral phenotype.

Example polymorphisms in linkage disequilibrium with any one or more of the 1,172 genes within the about 20 megabase region in chromosome CM010796.2 (i.e. between about 40 megabases and about 60 megabases), and which are associated with the day length neutral phenotype, are disclosed in Table 1. The skilled person understands that additional polymorphic sites not identified in Table 1 but that are within the about 20 megabase region, and the alleles present at these sites, may be used for the identification of plants that have the day length neutral phenotype or that are carriers of the trait for day length neutral phenotype. Thus, a further aspect of this disclosure is a method comprising testing nucleic acid from a Cannabis plant to determine the presence or absence of an allele at a polymorphic site as indicated, for example, for any one or more of the nucleotide sequences identified in Table 1. Again, the method is for identifying whether a Cannabis plant has a day length neutral phenotype, or is a carrier of a trait for a day length neutral phenotype. The presence of the allele indicates that the plant has a day length neutral phenotype or is a carrier of the trait for the day length neutral phenotype.

Example polymorphisms in linkage disequilibrium with the UPF2 allele comprising the deletion of SEQ ID NO: 2, and which are associated with the day length neutral phenotype, are disclosed in Tables 2, 3, and 4. The skilled person understands that additional polymorphic sites not identified in any of Tables 2, 3 or 4 but that are within approximately 3,000 bp upstream and 3,000 bp downstream of the Cannabis UPF2 gene, and the alleles present at these sites, may be used for the identification of plants that have the day length neutral phenotype or that are carriers of the trait for day length neutral phenotype. Thus, a further aspect of this disclosure is a method comprising testing nucleic acid from a Cannabis plant to determine the presence or absence of an allele at a polymorphic site as indicated, for example, for any one or more of the nucleotide sequences identified in Table 2, 3, or 4. Again, the method is for identifying whether a Cannabis plant has a day length neutral phenotype, or is a carrier of a trait for a day length neutral phenotype. The presence of the allele indicates that the plant has a day length neutral phenotype or is a carrier of the trait for the day length neutral phenotype.

The skilled person further understands that any polymorphic site that itself is in linkage disequilibrium with one or more of the example polymorphisms identified in Tables 1, 2, 3, and 4 that is associated with the day length neutral phenotype can also be used as marker for the day length neutral phenotype. Thus, a further aspect of this disclosure relates to a method comprising testing nucleic acid from a Cannabis plant to determine the presence or absence of a first allele that is in linkage disequilibrium r²=0.9 to 1 with a second allele at a polymorphic site as indicated for any one or more of the example nucleotide sequences identified in Table 1, 2, 3 or 4. The method is for identifying whether a Cannabis plant has a day length neutral phenotype, or is a carrier of a trait for a day length neutral phenotype. The presence of the second allele indicates that the plant has a day length neutral phenotype or is a carrier of the trait for the day length neutral phenotype, such that the presence of the first allele indicates that the plant has a day length neutral phenotype or is a carrier of the trait for the day length neutral phenotype.

In various embodiments of the methods described above, “testing” comprises nucleic acid amplification, e.g. amplification carried out by polymerase chain reaction (PCR).

The skilled person understands that testing may also involve sequencing, 5′ nuclease digestion, molecular beacon assay, oligonucleotide ligation assay, size analysis, single-stranded conformation polymorphism analysis, denaturing gradient gel electrophoresis (DGGE), or any other appropriate methodology as described elsewhere herein and in references cited herein.

In preferred embodiments, testing is performed using an allele-specific method, e.g. allele-specific probe hybridization, allele-specific primer extension, or allele-specific amplification. Testing may be carried out using an allele-specific primer that comprises a sequence selected from the group consisting of SEQ ID NOS: 3, 4 and sequences fully complementary thereto.

### Marker-Assisted Breeding

The skilled person further understands that the methods for identifying plants that have a day length neutral phenotype, or are a carrier of the trait, are particularly useful in methods of marker-assisted breeding to efficiently introduce the day length neutral trait into Cannabis varieties having desirable traits but a photoperiod phenotype.

Thus, another aspect of the disclosure relates to methods of producing a Cannabis plant with a day length neutral phenotype. The method comprises initially performing a first cross of a first parent having at least one allele associated with a day length neutral phenotype at a polymorphic site as indicated for any one of the example nucleotide sequences identified in Table 1, 2, 3, or 4 with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above under the section entitled “Identification of Plants that have an Autoflowering (“Day Length Neutral”) Phenotype”.

After a first progeny plant that is a carrier of a trait for a day length neutral phenotype is identified, then the first progeny plant may be self-pollinated to produce F₂ progeny. The method may then further include identifying an F₂ progeny plant that has a day length neutral phenotype according to the method described above. However, the skilled person would understand that 25% of the resulting F₂ progeny should have the day length neutral phenotype, such that the skilled person may choose not to screen for plants carrying two copies of the allele associated with the day length neutral phenotype. Alternatively, the skilled person may be interested in selecting F₂ progeny that are carriers of the trait and thus may choose an F₂ progeny plant that is a carrier of the trait for the day length neutral phenotype according to the method described above.

Alternatively, after a first progeny plant that is a carrier of a trait for a day length neutral phenotype is identified, the method may comprise backcrossing the first progeny plant to the first parent, and then identifying a progeny plant from the backcross that has a day length neutral phenotype according to a method described above. Such strategy may be of interest where the interest is in introducing a particular trait from the photoperiod sensitive variety to the variety with the day length neutral phenotype.

Alternatively, the method may comprise backcrossing the first progeny plant to the second parent, and then identifying a progeny plant from the backcross that is a carrier of the trait for the day length neutral phenotype according to the method as described above. Such strategy may be useful after a first progeny plant that is a carrier of a trait for a day length neutral phenotype is identified where the goal is to introduce only the autoflowering trait to the variety of the second parent.

In another embodiment, after a first progeny plant that is a carrier of a trait for a day length neutral phenotype is identified, the method further comprises performing a second cross of the first progeny plant with a second progeny plant identified as a carrier of a trait for a day length neutral phenotype according to a method as described above. The method then comprises identifying a progeny plant from the second cross that has a day length neutral phenotype, or is a carrier of the trait, according to a method as described above.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site as indicated for any one of the example nucleotide sequences identified in Table 1, 2, 3 or 4, with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In yet another aspect, the disclosure provides methods of producing a Cannabis plant with a day length neutral phenotype comprising initially identifying a first parent homozygous for at least one allele associated with a day length neutral phenotype at a polymorphic site as indicated for any one of the example nucleotide sequences identified in Table 1, 2, 3 or 4 according to a method described above. The method further comprises crossing the first parent to a second parent that has a day length sensitive phenotype to produce F₁ progeny. Again, the skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e. because 25% of the plants should have a day length neutral phenotype), the skilled person could do so if it were desirable to know the phenotype before flowering is initiated. Alternatively, the skilled person may want to screen the F₂ progeny for carriers of the trait.

The skilled person also understands that there may be many reasons as to why it may be beneficial to maintain lines that are heterozygous for the trait that causes the day length neutral phenotype. First, the phenotype is not completely recessive, such that plants heterozygous for the allele are somewhat sensitive to day length and thus initiate flowering later than plants having the day length neutral phenotype when grown under long days. Thus, there may be situations where it would be advantageous to select for and cultivate plants that had such an intermediate phenotype. Second, vegetative propagation of plants having a day length neutral phenotype may induce flowering. Accordingly, it may be advantageous in some circumstances to maintain the autoflowering trait in heterozygous plants and then perform the cross (or self-pollinate) to produce plants having the day length neutral phenotype when it is desired.

Presuming that the deletion of SEQ ID NO: 2 from the UPF2 gene is causative of the day length neutral phenotype, then the skilled person would understand that any plant having a loss or gain of function mutation in the UPF2 gene could be used in breeding programs to introduce the day length neutral phenotype into a day length sensitive variety. Presuming that a deletion from any one or more of the 1,172 genes within the about 20 megabase region is causative of the day length neutral phenotype, then the skilled person would understand that any plant having a loss or gain of function mutation in such gene(s) could be used in breeding programs to introduce the day length neutral phenotype into a day length sensitive variety. Thus, further aspects of the disclosure pertain to methods of producing a Cannabis plant with a day length neutral phenotype comprising initially performing a first cross of a first parent identified as having at least one loss of function mutation allele and/or at least one gain or function allele of any one or more of the 1,172 genes within the about 20 megabase region (for example, without limitation, at least one loss of function mutation allele and/or at least one gain of function mutation allele of the UPF2 gene) according to a method as described above with a second parent that has a day length sensitive phenotype. The method further comprises performing a second cross of a first progeny of the first cross with a second progeny of the first cross to produce a plant that is homozygous for the loss or gain of function mutation allele, which plant would have a day length sensitive phenotype.

### Polynucleotides and Kits

The skilled person further understands that the disclosure pertains to reagents and kits that are useful or necessary for performing the methods described above for identifying plants that have a day length neutral phenotype or are carriers of the trait for the day length neutral phenotype, and for marker assisted breeding of the day length neutral phenotype.

Thus, the disclosure further pertains to an allele-specific polynucleotide for use in the methods described herein. In some embodiments, the polynucleotide is specific for a variation (e.g. a deletion) in any one or more of the 1,172 endogenous genes within the about 20 megabase region. In particular embodiments, the polynucleotide is specific for the variation in the endogenous UPF2 gene. In particular embodiments, the polynucleotide is specific for the allele of UPF2 comprising the deletion of SEQ ID NO: 2. In particular embodiments, the allele-specific polynucleotide is specific for an allele at a polymorphic site as indicated for any one or more of the example nucleotide sequences identified in Table 1, 2, 3 or 4, or an allele that is in linkage disequilibrium therewith. The allele-specific polynucleotide may be at least 16 nucleotides in length. In various embodiments, the polynucleotide is detectably labeled, e.g. with a fluorescent dye.

The disclosure further pertains to kits for use in the methods described above. The kits comprise at least one allele-specific polynucleotide as described above and at least one further component, e.g. a buffer, deoxynucleotide triphosphates (dNTPs), an amplification primer pair, an enzyme, or any combination thereof. The enzyme may be a polymerase or a ligase.

In certain embodiments, the kits are provided to laboratories, whereby a breeder collects biological samples from individual plants and submits the samples to the laboratory for testing using the kits to determine the genotype at one or more polymorphic sites disclosed herein, and the plant's likelihood of having a day length neutral phenotype, and to provide the results to the breeder e.g. in the form of a report as described below. Alternatively, the kits include reagents for performing the testing of the samples by the breeders themselves.

The Applicant further understands that the methods disclosed herein could be automated and implemented with the use of computers. The results of a test to determine the identify of an allele at a polymorphic site may be presented as a “report”, that can be generated as part of a testing process, which can be provided to the breeder or any other intended recipient in physical or electronic form. Reports may include the alleles that the individual plant carries at various polymorphic sites and/or an individual plant's likelihood of having a day length neutral phenotype, or being a carrier of the trait.

### Molecular Modification

Presuming that a deletion from any one or more of the 1,172 genes within the about 20 megabase region is causative of the day length neutral phenotype, then the skilled person would understand that a Cannabis plant could be genetically modified to decrease expression of UPF2 gene to result in the day length neutral phenotype. For example, presuming that the deletion of SEQ ID NO: 2 from the UPF2 gene is causative of the day length neutral phenotype, then the skilled person would understand that a Cannabis plant could be genetically modified to decrease expression of UPF2 gene to result in the day length neutral phenotype.

Accordingly, a further aspect of the disclosure relates to methods for producing a day length neutral Cannabis plant comprising decreasing the expression of any one or more of the 1,172 genes within the about 20 megabase region (for example, without limitation, the endogenous UPF2 gene) in the plant. Decreasing the expression of the endogenous gene (s) (for example, without limitation, the endogenous UPF2 gene) may include introducing or producing a loss or gain of function allele in the endogenous gene. The loss or gain of function allele may include a point mutation, an insertion, or a deletion. The point mutation may be a substitution. The point mutation may be a nonsense mutation. An insertion could include a T-DNA or a transposable element. Decreasing the expression of the endogenous gene (s) (for example, without limitation, the endogenous UPF2 gene) may comprise expressing a heterologous nucleic acid molecule homologous to a portion of the endogenous gene, wherein the heterologous nucleic acid molecule decreases expression of the endogenous gene (s) (for example, without limitation, the endogenous UPF2 gene). The heterologous nucleic acid molecule may decrease expression of the gene(s) (for example, without limitation, the UPF2 gene) by RNA interference. In some embodiments, the heterologous nucleic acid molecule may comprise a portion of SEQ ID NO: 1.

A further aspect of the disclosure relates to methods of generating a Cannabis plant having a day length neutral phenotype comprising initially using a molecular methodology to identify a first plant as comprising a loss or gain of function allele in any one or more of the 1,172 genes within the about 20 megabase region (for example, without limitation, in the UPF2 gene). The method further comprises performing a first cross of said first plant to a second plant followed by performing a second cross of progeny from the first cross. Prior to performing the second cross, progeny from the first cross may be screened to identify plants comprising the loss or gain of function allele in any one or more of the 1,172 genes within the about 20 megabase region (for example, without limitation, the UPF2 gene) for use in the second cross. The method further comprises screening progeny of the second cross for a plant that is homozygous for the loss or gain of function allele in any one or more of the 1,172 endogenous genes within the about 20 megabase region (for example, without limitation, the endogenous UPF2 gene). The loss or gain of function allele in any one or more of the 1,172 endogenous genes within the about 20 megabase region (for example, without limitation, the endogenous UPF2 gene) may be generated by genetic modification of the first plant or an ancestor thereof. The molecular methodology may include targeting induced local lesions in genomes (TILLING) methodology.

### Reduction of Gene Expression

Gene expression and/or activity in genetically modified plants of the present invention may be reduced by any method that results in reduced activity of the corresponding protein in the plant. For example, UPF2 gene expression and/or activity in genetically modified plants of the present invention may be reduced by any method that results in reduced activity of the UPF2 protein in the plant. This may be achieved by e.g. by altering gene activity (for example, without limitation, UPF2 gene activity) at the DNA, mRNA and/or protein levels.

### Mutating the Endogenous Genes

In one aspect, the present disclosure relates to genetic modifications targeting one or more of the 1,172 endogenous genes within the about 20 megabase region (for example, without limitation, the UPF2 gene) to alter gene expression and/or activity. The endogenous gene(s) (for example, without limitation, the endogenous UPF2 gene) may be altered by, without limitation, knocking-out the gene(s) (e.g. the UPF2 gene); or knocking-in a heterologous DNA to disrupt the gene(s) (for example, without limitation, the UPF2 gene). The skilled person would understand that these approaches may be applied to the coding sequences, the promoter or other regulatory elements necessary for gene transcription. For example, technologies such as CRISPR/Cas9 and TALENS can be used to introduce loss or gain of function mutations in one or more of the 1,172 endogenous genes within the about 20 megabase region (for example, without limitation, the endogenous UPF2 gene). Plants having at least one mutagenized allele comprising a loss or gain of function mutation can then be self-fertilized to produce progeny homozygous for the loss or gain of function alleles in one or more of the 1,172 genes within the about 20 megabase region (for example, without limitation, the UPF2 gene).

Deletions lack one or more nucleotides of the one or more of the 1,172 endogenous genes within the about 20 megabase region (e.g. the endogenous UPF2 gene) or residues of the corresponding endogenous protein (e.g. the UPF2 protein). For the purposes of this disclosure, a deletion variant includes embodiments in which no amino acids of the endogenous protein are translated, e.g. where the initial “start” methionine is substituted or deleted.

Insertional mutations typically involve the addition of material at a non-terminal point in the gene or polypeptide, but may include fusion proteins comprising amino terminal and carboxy terminal additions. Substitutional variants typically involve a substitution of one amino acid for another at one or more sites within the protein.

### Expression of Transgenes Targeting the Endogenous Genes

In another aspect, the present disclosure relates to reducing the expression and/or activity of one or more of the 1,172 genes within the about 20 megabase region (e.g. the endogenous UPF2 gene) by targeting its mRNA transcripts. In this regard, levels of corresponding mRNA transcripts (e.g. levels of UPF2 mRNA transcripts) may be reduced by methods known in the art including, but not limited to, co-suppression, antisense expression, short hairpin (shRNA) expression, interfering RNA (RNAi) expression, double stranded (dsRNA) expression, inverted repeat dsRNA expression, micro interfering RNA (miRNA), simultaneous expression of sense and antisense sequences, or a combination thereof. Various such methods are described in WO2018/027324.

In one embodiment, the present disclosure relates to the use of nucleic acid molecules that are complementary, or essentially complementary, to at least a portion of the molecule set forth in SEQ ID NO: 1. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO: 1 under physiological conditions.

The phenomenon of co-suppression in plants relates to the introduction of transgenic copies of a gene resulting in reduced expression of the transgene as well as the endogenous gene. The observed effect depends on sequence identity between the transgene and the endogenous gene.

The term “RNA interference” (RNAi) refers to well-known methods for down regulating or silencing expression of a naturally occurring gene in a host plant. RNAi employs a double-stranded RNA molecule or a short hairpin RNA to change the expression of a nucleic acid sequence with which they share substantial or total homology.

Antisense suppression of gene expression does not involve the catalysis of mRNA degradation, but instead involves single-stranded RNA fragments binding to mRNA and blocking protein translation.

Expression of one or more of the 1,172 genes within the about 20 megabase region (for example, without limitation, expression of the UPF2 gene) may be suppressed using a synthetic gene(s) or an unrelated gene(s) that contains about 21 bp regions or longer of high homology (preferably 100% homology) to the endogenous coding sequences of the gene (for example, without limitation, the UPF2 gene).

Nucleic acid molecules that are substantially identical to portions of one or more of the 1,172 genes within the about 20 megabase region (for example, without limitation, to portion of the UPF2 gene) may also be used in the context of the disclosure. As used herein, one nucleic acid molecule may be “substantially identical” to another if the two molecules have at least 60%, at least 70%, at least 80%, at least 82.5%, at least 85%, at least 87.5%, at least 90%, at least 92.5%, at least 95%, at least 96%, at least 97%, at least 98%, or at least 99% sequence identity. Thus, a nucleic acid sequence comprising a nucleic acid sequence that is at least 60%, at least 65%, at least 70%, at least 75%, at least 80%, at least 85%, at least 90%, at least 92.5%, at least 95%, at least 96%, at least 97%, at least 98% or at least 99% identical to SEQ ID NO: 1 may be suitable for use in the context of this disclosure.

Fragments of nucleic acid sequences of one or more of the 1,172 genes within the about 20 megabase region (for example, without limitation, the UPF2 gene) may be used. Such fragments may have lengths of at least 20, at least 50, at least 100, at least 150, at least 200, at least 250, at least 300, at least 350, at least 400, at least 450 or at least 500 contiguous nucleotides of a nucleic acid sequence of the gene (for example, without limitation, the UPF2 gene) as the case may be.

In one embodiment, a genetically modified Cannabis plant of the disclosure comprises, stably integrated into its genome a nucleic acid molecule heterologous to the plant. The nucleic acid molecule encodes an RNA, e.g. a hairpin RNA, for reducing expression of one or more of the 1,172 genes within the about 20 megabase region (for example, without limitation, the UPF2 gene). The nucleic acid molecule may be arranged in the sense orientation relative to a promoter. In another embodiment, the nucleic acid molecule may be arranged in the anti-sense orientation relative to a promoter. In a further embodiment, a genetic construct may comprise at least two nucleic acid molecules in both the sense and anti-sense orientations, relative to a promoter. A genetic construct comprising nucleic acids in both the sense and anti-sense orientations may result in mRNA transcripts capable of forming stem-loop (hairpin) structures.

In various instances, the nucleic acid molecule comprises at least 20, at least 50, at least 100, at least 150, at least 200, at least 250, at least 300, at least 350, at least 400, at least 450 or at least 500 contiguous nucleotides of a nucleic acid sequence possessing at least 80%, at least 90% or 100% sequence identity to the nucleic acid sequence set forth in SEQ ID NO: 1,

The skilled person will also readily understand that although in the foregoing illustrative examples partial gene sequences (e.g. partial UPF2 gene sequences) were suggested for constructing the constructs, complete coding sequences, alternative coding sequences, 5′UTR and/or 3′UTR, or mutated derivatives of these sequences can also be used. The maximum number of nucleic acid molecules that may be used in the context of the invention may be limited only by the maximum size of the construct that may be delivered to a target plant or plant cell using a given transformation method.

The skilled person would also appreciate that a nucleic acid molecule comprising the sequence of a gene promoter and/or other regulatory elements may be used in the context of the invention. For example, a nucleic acid molecular comprising the sequence of a UPF2 gene promoter and/or other regulatory elements may be used in the context of the invention. In an embodiment, a heterologous nucleic acid molecule comprising sequences of a UPF2 gene promoter and/or regulatory element may be used to bias the cellular machinery away from an endogenous UPF2 gene promoter thus resulting in reduced UPF2 expression.

### Plant Transformation

The introduction of DNA into plant cells by Agrobacterium mediated transfer is well known to those skilled in the art. Use of Agrobacterium transformation to transform Cannabis plants has been described, for example, in US patent application publication no. 2019/0085347A1.

Nevertheless, the present invention is not limited to any method for transforming plant cells, and the skilled person will readily understand that any other suitable method of DNA transfer into plant cells may be used. Methods for introducing nucleic acids into cells (also referred to herein as “transformation”) are known in the art as described in WO2018/027324.

Another method for introducing DNA into plant cells is by biolistics, which involves the bombardment of plant cells with microscopic particles (such as gold or tungsten particles) coated with DNA. The particles are rapidly accelerated, typically by gas or electrical discharge, through the cell wall and membranes, whereby the DNA is released into the cell and incorporated into the genome of the cell. This method is used for transformation of many crops, including corn, wheat, barley, rice, woody tree species and others.

Another method for introducing DNA into plant cells is by electroporation. This method involves a pulse of high voltage applied to protoplasts/cells/tissues resulting in transient pores in the plasma membrane which facilitates the uptake of foreign DNA.

Plant cells may also be transformed by liposome mediated gene transfer.

The nucleic acid constructs of the present invention may be introduced into plant protoplasts, which are cells in which its cell wall is completely or partially removed and then transformed with known methods.

A nucleic acid molecule of the present invention may also be targeted into the genome of a plant cell by a number of methods including, but not limited to, targeting recombination, homologous recombination and site-specific recombination. Methods of homologous recombination and gene targeting in plants are known in the art.

As used herein, “targeted recombination” refers to integration of a nucleic acid construct into a site on the genome, where the integration is facilitated by a construct comprising sequences corresponding to the site of integration.

Homologous recombination relies on sequence identity between a piece of DNA that is introduced into a cell and the cell's genome. Homologous recombination is an extremely rare event in higher eukaryotes. However, the frequency of homologous recombination may be increased with strategies involving the introduction of DNA double-strand breaks, triplex forming oligonucleotides or adeno-associated virus.

“Site-specific recombination” as used herein refers to the enzymatic recombination that occurs when at least two discrete DNA sequences interact to combine into a single nucleic acid sequence in the presence of an enzyme. Enzymes and systems that have been developed to induce targeted mutagenesis and targeted deletions include Zinc Finger Nucleases (ZFNs), Meganucleases, Transcription Activator-Like Effector Nucleases (TALENS), and Clustered Regularly Interspaced Short Palindromic Repeats (CRISPR)-associated nucleases (e.g. CRISPR/Cas9,

In one embodiment, the polynucleotide encodes a zinc finger protein that binds to one or more of the 1,172 genes within the about 20 megabase region (e.g. the UPF2 gene), resulting in reduced expression of the gene. In particular embodiments, the zinc finger protein binds to a regulatory region of the gene (e.g. a regulatory region of the UPF2 gene). In other embodiments, the zinc finger protein binds to mRNA (e.g. UPF2 mRNA) to prevent its translation. Methods of selecting sites for targeting by zinc finger proteins have been described, for example, in U.S. Pat. No. 6,453,242, and methods for using zinc finger proteins to inhibit the expression of genes in plants are described, for example, in US2003/0037355.

The nucleic acid molecule becomes stably integrated into the plant genome such that it is heritable to daughter cells in order that successive generations of plant cells have reduced gene expression. This may involve the nucleic acid molecules of the present invention integrating, for instance randomly, into the plant cell genome. Alternatively, the nucleic acid molecules of the present invention may remain as exogenous, self-replicating DNA that is heritable to daughter cells. As used herein, heterologous, self-replicating DNA that is heritable to daughter cells is also considered to be “stably integrated into the plant genome”.

Disruption of an endogenous gene (for example, without limitation, the endogenous UPF2 gene) may be confirmed by methods known in the art of molecular biology. For example, disruption of endogenous genes may be assessed by PCR followed by Southern blot analysis. mRNA levels may, for example, be measured by real time PCR, RT-PCR, Northern blot analysis, micro-array gene analysis, and RNAse protection.

### Targeted Screening for Loss or Gain of Function Gene Mutations

This disclosure further relates to methods of generating Cannabis plants with day length neutral phenotype that involve targeted screening for loss or gain of function mutations in one or more of the 1,172 genes within the about 20 megabase region (for example, without limitation, the endogenous UPF2 gene) and subsequent breeding of plants to obtain plants homozygous for the loss or gain of function mutation at the locus. Cannabis breeders have used a variety of selection techniques in the development of improved cultivars. However, the most successful breeding method involves the hybridization of parents with a variety of different desired characteristics.

The term “T-DNA insertion” refers to methods utilizing transfer-DNA (T-DNA) for disrupting genes via insertional mutagenesis. Down-regulating or silencing expression of the endogenous gene (for example, without limitation, the endogenous UPF2 gene) could be achieved by T-DNA mutagenesis, wherein the T-DNA is randomly inserted in the plant genome to introduce mutations. Subsequently, plants can be screened for T-DNA insertions in the gene.

Mutations (including deletions, insertions, and point mutations) can also be introduced randomly into the genome of a plant cell by various forms of mutagenesis to produce non-natural variants. Methods for mutagenesis of plant material, including seeds, and subsequent screening or selection for desired phenotypes are well known, as described in WO2009/109012. Mutagenized plants and plant cells can also be specifically screened for mutations in any one of the 1,172 genes within the about 20 megabase region (for example, without limitation, the UPF2 gene), for example, by TILLING (Targeting Induced Local Genomes). Loss or gain of function mutations present in natural plant populations can be identified by EcoTILLING.

Once the loss or gain of function mutation in the endogenous gene has been identified, traditional breeding processes can be used to produce plants homozygous for the loss or gain of function mutations at the endogenous gene.

### Plants Having an Autoflowering (“Day Length Neutral”) Phenotype

The skilled person further understands that aspects of this disclosure pertain to novel plants having a day length neutral phenotype, or that are carriers of the day length neutral phenotype.

In one aspect, the disclosure pertains to a Cannabis plant or plant cell generated according to a method as described above in the section entitled “Marker-assisted Breeding” and “Molecular Modification”.

In another aspect, the disclosure pertains to a genetically modified Cannabis plant or plant cell having a photoperiod phenotype, wherein the Cannabis plant or plant cell is genetically modified to have reduced expression of one or more of the 1,172 endogenous genes within the about 20 megabase region (for example, without limitation, reduced expression of the endogenous UPF2 gene). In various embodiments, the plant or plant cell comprises an expression construct for reducing the expression of the endogenous gene (for example, without limitation, the endogenous UPF2 gene). The expression construct comprises a nucleic acid molecule encoding a hairpin RNA for reducing expression of the endogenous gene (for example, without limitation, the endogenous UPF2 gene). For example, the endogenous UPF2 gene may have the sequence of SEQ ID NO: 1 or be at least 95% identical to SEQ ID NO: 1. The nucleic acid molecule encoding the hairpin RNA may comprises a portion of SEQ ID NO: 1. In some embodiments, the plant or plant cell is a seed or seed cell.

In various aspects, the disclosure pertains to seeds produced by a Cannabis plant or plant cell as described above. In various aspects, the disclosure pertains to a method of producing seeds comprising growing a Cannabis plant as described above, allowing the Cannabis plant to flower, be pollinated, and set seed, and harvesting the seed from the plant.

In various aspects, the disclosure pertains to dried flower from a Cannabis plant having a day length neutral phenotype as described above, or comprising a Cannabis plant cell as described above.

In various aspects, the disclosure relates to a crop comprising a plurality of Cannabis plants as described above.

In various aspects, the disclosure relates to expression vectors for generating a day length neutral Cannabis plant, the expression vector comprising a nucleic acid comprising a portion of SEQ ID NO. 1.

In various aspects, the disclosure relates to use of a polynucleotide molecule having a sequence comprising a portion of SEQ ID NO: 1 for generating a Cannabis plant with a day length neutral phenotype.

In various aspects, the disclosure relates to use of a plant or plant cell as described above for the production of cannabinoids and/or terpenes.

### Value Added Products

The skilled person understands that the disclosure further pertains to cannabinoids and/or terpenes derived from a plant or plant cell as described above, or extracted from dried flower as described above. Thus, the disclosure further pertains to methods of producing cannabinoids and/or terpenes comprising extracting cannabinoids and/or terpenes from flowers harvested from plants as described above.

The skilled person further understands that the plants and plant cells described above could further be used to derive extracts, concentrates, isolates, and oils containing cannabinoids and/or terpenes, which may be consumed or optionally used in the production of a variety of food items. The food items may be edible oils. The food items may be snack foods, e.g. candy. The candy may be chocolate, gummies, mints, lozenges, lollipops, or chewing gums. The food items may be baked goods, including, but not limited to, cookies, brownies, cakes, or breads. The food items may be beverages including, but not limited to, soft drinks, energy drinks, teas, coffees, juices, or waters.

The skilled person further understands that the plants and plant cells described above could further be used to derive extracts, concentrates, isolates, and oils containing cannabinoids and/or terpenes, which may be used in the production of cosmetics and/or topicals. The topicals may be massage creams, massage oils, bath oils, body oils, cosmetic oils, oils for toiletry purposes, skin creams, skin lotions, lip care preparations, face and body lotions, bath additives, soaps for personal use, beauty creams for body care, non-medicated skin preparations, or medicated skin preparations.

The disclosure further pertains to extracts, concentrates, isolates, and oils containing cannabinoids and/or terpenes derived from a Cannabis plant or plant cell, or dried flower, as described above, and pharmaceutical compositions comprising such extracts, concentrates, isolates, and oils, wherein the extracts, concentrates, isolates, and oils contain cannabinoids and/or terpenes.

The disclosure further pertains to methods of producing oils containing cannabinoids and/or terpenes, the methods comprising crushing seeds produced by a plant described above.

The disclosure further pertains to methods of producing oils, extracts, and concentrates containing cannabinoids and/or terpenes, the methods comprising extracting the oils, extracts, and concentrates from a plant or plant cell, or dried flower, as described above.

The disclosure further pertains to methods of producing isolates comprising a cannabinoid or terpene, the methods comprising isolating the cannabinoid or terpene from a plant or plant cell, or dried flower, as described above.

While specific embodiments of the invention have been described and illustrated, such embodiments should be considered illustrative of the invention only and not as limiting the invention as construed in accordance with the accompanying claims.

All applications and publications referred to herein are incorporated by reference in their entirety.

TABLE 1
Polymorphic sites located within a 20 megabase
region of the Cannabis chromosome CM010796.2
located between about 40 megabases to about
60 megabases.

| Poly- | | | | Genomic | SEQ |
|---|---|---|---|---|---|
| morphism | Chromo- | | | Context | ID |
| ID | some | Haplotype | Variant | Sequence | NO: |
| SCM010796.2_ | CM010 | GGAA | GAGAA | GCTACTGGAA | 5 |
| 55541136 | 796.2 | AAGG | AAGAA | ACCTGTCTAT | |
| | | AA/G | | TTTCTAGTGC | |
| | | AGAA | | AATGAAGTAT | |
| | | AAGA | | AATATGGACA | |
| | | A | | ATGAGGCAAG | |
| | | | | GCAATGTAAG | |
| | | | | TAGGTTAAGA | |
| | | | | GATAAAAGTT | |
| | | | | TCAAGGCACT | |
| | | | | GGAAAAGGAA | |
| | | | | TGGAAAGTAT | |
| | | | | AATCATAGTT | |
| | | | | AGGCAGAGAA | |
| | | | | AGATTTGAAC | |
| | | | | TGTTATACGG | |
| | | | | TTCTTTTATG | |
| | | | | GGATGGGAGT | |
| | | | | TTATTATGAA | |
| | | | | TTTCAAGTGG | |
| | | | | AGAAAAAAAA | |
| SCM010796.2_ | CM010 | G/T | T | AACACTCAAC | 6 |
| 56140841 | 796.2 | | | ACACAAATAT | |
| | | | | ACAAATATCA | |
| | | | | AATACACATA | |
| | | | | CACTCACACA | |
| | | | | CACAAATGGT | |
| | | | | AATGAAAGAA | |
| | | | | AGAAGACGGT | |
| | | | | ACCTTAAAAG | |
| | | | | CAGAACGAGC | |
| | | | | GACATAGCCC | |
| | | | | AGGCGTTGAG | |
| | | | | CTTCTCTGTA | |
| | | | | GAAAAAATCT | |
| | | | | GGAGCTCCAG | |
| | | | | CTCCACTCAT | |
| | | | | CTCTGCAAAT | |
| | | | | GTTAAAACCC | |
| | | | | TAAACCCCGG | |
| | | | | TAGTCGCACA | |
| | | | | G | |
| SCM010796.2_ | CM010 | T/A | A | CTGGCGGTGC | 7 |
| 57309299 | 796.2 | | | CCAAAATTTT | |
| | | | | GGGCACCCGG | |
| | | | | TTGAAAATTT | |
| | | | | GATTGCCTGG | |
| | | | | TTGGGAACIT | |
| | | | | TTTCGAGTAT | |
| | | | | CTCAATTTTT | |
| | | | | GTGATATTTA | |
| | | | | GGATATTGAC | |
| | | | | TCTTCATTTA | |
| | | | | TGGATGGAAA | |
| | | | | AATTTTAAAT | |
| | | | | TATAAATTTA | |
| | | | | AGTTGTAAAA | |
| | | | | AGTATTTTAG | |
| | | | | TGAGACATAT | |
| | | | | ACCAATTTAC | |
| | | | | CATTAAATAT | |
| | | | | TTTCTACTAC | |
| | | | | T | |
| SCM010796.2_ | CM010 | G/A | A | GTTATATGGA | 8 |
| 50858909 | 796.2 | | | GACTTCACAT | |
| | | | | CCACCACTAA | |
| | | | | CAACGTGGCC | |
| | | | | ATGATGGTGG | |
| | | | | TTTACCTTGG | |
| | | | | GCTAGTGCTG | |
| | | | | CTAGTGAGTG | |
| | | | | GCAGTTTGAT | |
| | | | | CTTGCCACAT | |
| | | | | GGAGTAATGG | |
| | | | | CAGCTCCACT | |
| | | | | GAAACCATAC | |
| | | | | ATTAGATGTT | |
| | | | | CGCATGGATT | |
| | | | | TAGGTGTTTG | |
| | | | | AGTGACAAAC | |
| | | | | CAATCTGGAT | |
| | | | | TAAAGTTTGC | |
| | | | | TTGAATAAGA | |
| | | | | T | |
| SCM010796.2_ | CM010 | A/G | G | CCTCCTTGTT | 9 |
| 44181271 | 796.2 | | | TTTACATAAT | |
| | | | | CAATGCATGC | |
| | | | | CTTATTCTTC | |
| | | | | TTTTACTTTA | |
| | | | | TCTTTATTTT | |
| | | | | TTTTTCTTTA | |
| | | | | AAATTACATT | |
| | | | | TTCTTTAGTT | |
| | | | | TATTGGTGAT | |
| | | | | ATCCCTTCTA | |
| | | | | GCTTAGCATA | |
| | | | | ATTTTGCCTG | |
| | | | | TACTATTAAG | |
| | | | | GCAATCAAAT | |
| | | | | TTTGTCTTCA | |
| | | | | TTTTTACAAT | |
| | | | | TTTAGTATTT | |
| | | | | TCTTGTACTG | |
| | | | | AATAAGATTT | |
| | | | | T | |
| SCM010796.2_ | CM010 | C/T | T | GTGTCTCCCT | 10 |
| 40320374 | 796.2 | | | AAATCCCGTG | |
| | | | | GGAGCCTTGG | |
| | | | | CTTCCGAAAA | |
| | | | | ACTAAGAAAA | |
| | | | | TGAATCAGGA | |
| | | | | TTTCTTGGCT | |
| | | | | AAGTAGGCTT | |
| | | | | GGAAACTGCT | |
| | | | | TACTAAATCC | |
| | | | | CAATCTTTGT | |
| | | | | GTTGAAGAAT | |
| | | | | TCTAAGAGCT | |
| | | | | AAATACCTTA | |
| | | | | GAGGAAAGGA | |
| | | | | TATGCTTAAG | |
| | | | | TGTAAGGCTA | |
| | | | | AGTCTGTTGA | |
| | | | | CTTTTGGTTT | |
| | | | | TAGAAAAGTG | |
| | | | | T | |
| SCM010796.2_ | CM010 | A/G | G | CCTTCAGATT | 11 |
| 59999985 | 796.2 | | | ATAATACAAA | |
| | | | | ACACAATAAG | |
| | | | | AAATTTGAAA | |
| | | | | ATACAGATAA | |
| | | | | AGAAGCAATA | |
| | | | | TATAACCATG | |
| | | | | TGGAACTGTT | |
| | | | | CAAGTATGTC | |
| | | | | TACGCAATCA | |
| | | | | ACATTACCGG | |
| | | | | ATTTCCATCA | |
| | | | | GAATCTCCAA | |
| | | | | AGCCTTTTTT | |
| | | | | GGGACCATAT | |
| | | | | TAATAGATTG | |
| | | | | CAACTGAAAA | |
| | | | | TTTCCATGTC | |
| | | | | AACATTAGTA | |
| | | | | GAAGAAGATC | |
| | | | | A | |
| SCM010796.2_ | CM010 | G/A | A | TTTAAATGAG | 12 |
| 54639687 | 796.2 | | | TAATTTAATT | |
| | | | | TCATGTTATT | |
| | | | | ATACGTAAAA | |
| | | | | AAAAATATAT | |
| | | | | GTATATATAT | |
| | | | | GAAAATTAAC | |
| | | | | ATTAAAATTT | |
| | | | | TACTTTTTAT | |
| | | | | TTTTGGATTA | |
| | | | | GATTTATCCA | |
| | | | | ATTCAATCCA | |
| | | | | ATAAATACTA | |
| | | | | GATCTAAATT | |
| | | | | ATTGGATGAA | |
| | | | | TCTAATTCGA | |
| | | | | TCCATAAATA | |
| | | | | TTAGGTGTAA | |
| | | | | AATATTAGAT | |
| | | | | AAACTTGAAT | |
| | | | | T | |
| SCM010796.2_ | CM010 | T/C | C | ACTGGTTTAA | 13 |
| 51022534 | 796.2 | | | TATATAAATA | |
| | | | | TATAAAATAA | |
| | | | | TATAAAATTT | |
| | | | | AAAAGAAGAA | |
| | | | | GAATATAAAG | |
| | | | | AGAGAAAAAA | |
| | | | | ATTAAATGAA | |
| | | | | AATTTTTCAG | |
| | | | | TTAGAGTGAA | |
| | | | | TTCTTATTTA | |
| | | | | TACAAAAATA | |
| | | | | TGATTAAAAG | |
| | | | | AATGAAAAAT | |
| | | | | TAAACTAATG | |
| | | | | TAATAAAGAA | |
| | | | | AAATACAACT | |
| | | | | AAGCATTAAT | |
| | | | | GGCGATAATT | |
| | | | | AATTTGGTGA | |
| | | | | T | |
| SCM010796.2_ | CM010 | A/T | T | CTAAGTTTGA | 14 |
| 48159974 | 796.2 | | | GATGAAGGAC | |
| | | | | TTAGGAAATG | |
| | | | | CCACTTAAAT | |
| | | | | CCTTGGAATA | |
| | | | | ATTATTGTGA | |
| | | | | GGGATAAAGG | |
| | | | | CAAGGGATCC | |
| | | | | TAAAGATGTG | |
| | | | | TCAGGAAGAT | |
| | | | | AACATTCAGA | |
| | | | | AGGTGATTGA | |
| | | | | GAAGTGTTGG | |
| | | | | GAATATTTTA | |
| | | | | CTAGGATCT | |
| | | | | AGATTTACTA | |
| | | | | ACAAGTATGA | |
| | | | | TGATTAACAT | |
| | | | | CCTAAATATG | |
| | | | | AATATCTCTA | |
| | | | | AA | |

TABLE 2
Polymorphic sites located within +/−10 kb
region of the Cannabis UPF2 gene.

| | | | | | SEQ |
|---|---|---|---|---|---|
| Polymorphism | | | | Genomic Context | ID |
| ID | Chromosome | Haplotype | Variant | Sequence | NO: |
| SCM010796.2_ | CM010796.2 | AATGTGTTGTA | AATGTGTTGTAA | AATATGCATTTAGAATGGTT | 15 |
| 0 | | ATGGTACTCAT | TGGTACTCATCG | GAGAGTTTATGATAATGATA | |
| | | CGTTTTAGTTG | TTTTAGTTGCCC | ATGCAATACCAATTTCATAT | |
| | | CCCCACGGCT | CACGGCTACAA | GACCAATTCCTCTATTGTAT | |
| | | ACAAATGTTTT | ATGTTTTCAAAT | CCATATTGTTTTGGACAGTT | |
| | | CAAATCGTTTC | CGTTCGAGTTCC | AATGTGTTGTAATGGTACTC | |
| | | GAGTCCAATT | AATTGGGTAAG | ATCGTTTTAGTTGCCCCACG | |
| | | GGGTAAGGTG | GTGTGGTAATTA | GCTACAAATGTTTTCAAATC | |
| | | TGGTAATTAA | AAACAAATCTAT | GTTTCGAGTCCAATTGGGTA | |
| | | AACAAATCTAT | AATGATAATGTA | AGGTGTGGTAATTAAAACAA | |
| | | AATGATAATG | ATACCAACCTAT | ATCTATAATGATAATGTAAT | |
| | | TAATACCAACC | ACGACCAATTCT | ACCAACCTTATACGACCAAT | |
| | | TTATACGACCA | TGTATGGTATCC | TCTTGTATGGTATCCATATT | |
| | | ATTCTTGTATG | ATATTGTTTTGA | GTTTTGGACAGTTGTTTTTT | |
| | | GTATCCATATT | CAGTT | TTTTTTCTTGTAATGGTACT | |
| | | GTTTTGGACA | | CACCGTTTCAGTTGTCCAAT | |
| | | GTT/ | | GTTTTCAAATCGTGTTTTGA | |
| | | AATGTGTTGTA | | GTCCAATTGGAAAAGGTGTG | |
| | | | | GTAGTTTTAATAA | |
| | | ATGGTACTCAT | | | |
| | | CGTTTTAGTTG | | | |
| | | CCCCACGGCT | | | |
| | | ACAAATGTTTT | | | |
| | | CAAATCGTTCG | | | |
| | | AGTTCCAATTG | | | |
| | | GGTAAGGTGT | | | |
| | | GGTAATTAAA | | | |
| | | ACAAATaATA | | | |
| | | ATGATAATGT | | | |
| | | AATACCAACCT | | | |
| | | ATACGACCAA | | | |
| | | TTCTTGTATGG | | | |
| | | TATCCATATTG | | | |
| | | TTTTGACAGTT | | | |
| SCM010796.2_ | CM010796.2 | AGTCC/AGTCA | AGTCA | ATTGATTATAAAATAAATAG | 16 |
| 1 | | | | AGCTTAAGAAACAAAAAAAT | |
| | | | | TAAAGAAGAAGAAAACTAAG | |
| | | | | ATTTTTACGTGGTTGGAGCG | |
| | | | | TTGATGAACTTTAGTCCACG | |
| | | | | AGTCCATATATTATTAATTT | |
| | | | | GAGAAACTTTGATGTTTTAC | |
| | | | | ACAAGGATAATATTTTTTCC | |
| | | | | AAACTTAAGAACCCTAATTT | |
| | | | | AAGTCTTAGTATTTGAATAA | |
| | | | | T | |
| SCM010796.2_ | CM010796.2 | ATTAA/ATAA/ | ATAA/GTTAG/ | ATAAATAGAGCTTAAGAAAC | 17 |
| 2 | | GTTAG/ATTAG | ATTAG | AAAAAAATTAAAGAAGAAGA | |
| | | | | AAACTAAGATTTTTACGTGG | |
| | | | | TTGGAGCGTTGATGAACTTT | |
| | | | | AGTCCACGAGTCCATATATT | |
| | | | | ATTAATTTGAGAAACTTTGA | |
| | | | | TGTTTTACACAAGGATAATA | |
| | | | | TTTTTTCCAAACTTAAGAAC | |
| | | | | CCTAATTTAAGTCTTAGTAT | |
| | | | | TTGAATAATGATTTTCCACC | |
| | | | | A | |
| SCM010796.2_ | CM010796.2 | T/C | C | AAAATTAAAGAAGAAGAAAA | 18 |
| 3 | | | | CTAAGATTTTTACGTGGTTG | |
| | | | | GAGCGTTGATGAACTTTAGT | |
| | | | | CCACGAGTCCATATATTATT | |
| | | | | AATTTGAGAAACTTTGATGT | |
| | | | | TTTACACAAGGATAATATTT | |
| | | | | TTTCCAAACTTAAGAACCCT | |
| | | | | AATTTAAGTCTTAGTATTTG | |
| | | | | AATAATGATTTTCCACCATT | |
| | | | | TGCATGAAGATACAATCTTC | |
| | | | | T | |
| SCM010796.2_ | CM010796.2 | T/G | G | AAGATTTTTACGTGGTTGGA | 19 |
| 4 | | | | GCGTTGATGAACTTTAGTCC | |
| | | | | ACGAGTCCATATATTATTAA | |
| | | | | TTTGAGAAACTTTGATGTTT | |
| | | | | TACACAAGGATAATATTTTT | |
| | | | | TCCAAACTTAAGAACCCTAA | |
| | | | | TTTAAGTCTTAGTATTTGAA | |
| | | | | TAATGATTTTCCACCATTTG | |
| | | | | CATGAAGATACAATCTTCTA | |
| | | | | TTATAGGCTAGGGTTAAGAC | |
| | | | | A | |
| SCM010796.2_ | CM010796.2 | A/G | G | GGTTGGAGCGTTGATGAACT | 20 |
| 5 | | | | TTAGTCCACGAGTCCATATA | |
| | | | | TTATTAATTTGAGAAACTTT | |
| | | | | GATGTTTTACACAAGGATAA | |
| | | | | TATTTTTTCCAAACTTAAGA | |
| | | | | ACCCTAATTTAAGTCTTAGT | |
| | | | | ATTTGAATAATGATTTTCCA | |
| | | | | CCATTTGCATGAAGATACAA | |
| | | | | TCTTCTATTATAGGCTAGGG | |
| | | | | TTAAGACAGTTAGGATTTTT | |
| | | | | G | |
| SCM010796.2_ | CM010796.2 | T/A | A | GTTTTACACAAGGATAATAT | 21 |
| 6 | | | | TTTTTCCAAACTTAAGAACC | |
| | | | | CTAATTTAAGTCTTAGTATT | |
| | | | | TGAATAATGATTTTCCACCA | |
| | | | | TTTGCATGAAGATACAATCT | |
| | | | | TCTATTATAGGCTAGGGTTA | |
| | | | | AGACAGTTAGGATTTTTGTA | |
| | | | | TGAAGCTAAACCCACTGAAG | |
| | | | | GAGTAAATACATTTAATAGT | |
| | | | | GACTTTAGATTAATTAGAAA | |
| | | | | A | |
| SCM010796.2_ | CM010796.2 | ATTATAGGCTA | ATTTATAGGCTAG | TTACACAAGGATAATATTTT | 22 |
| 7 | | GG/ATTTATAGG | G/ATTTAGCCGCT | TTCCAAACTTAAGAACCCTA | |
| | | CTAGG/ATTTAG | AA/ATTTATACGC | ATTTAAGTCTTAGTATTTGA | |
| | | CCGCTAA/ATTT | TAGG | ATAATGATTTTCCACCATTT | |
| | | ATACGCTAGG | | GCATGAAGATACAATCTTCT | |
| | | | | ATTATAGGCTAGGGTTAAGA | |
| | | | | CAGTTAGGATTTTTGTATGA | |
| | | | | AGCTAAACCCACTGAAGGAG | |
| | | | | TAAATACATTTAATAGTGAC | |
| | | | | TTTAGATTAATTAGAAAAGA | |
| | | | | A | |
| SCM010796.2_ | CM010796.2 | G/T | T | ACTTAAGAACCCTAATTTAA | 23 |
| 8 | | | | GTCTTAGTATTTGAATAATG | |
| | | | | ATTTTCCACCATTTGCATGA | |
| | | | | AGATACAATCTTCTATTATA | |
| | | | | GGCTAGGGTTAAGACAGTTA | |
| | | | | GGATTTTTGTATGAAGCTAA | |
| | | | | ACCCACTGAAGGAGTAAATA | |
| | | | | CATTTAATAGTGACTTTAGA | |
| | | | | TTAATTAGAAAAGAACTCAT | |
| | | | | TTACAAGATAACGATCATCT | |
| | | | | A | |
| SCM010796.2_ | CM010796.2 | G/A | A | CTTAAGAACCCTAATTTAAG | 24 |
| 9 | | | | TCTTAGTATTTGAATAATGA | |
| | | | | TTTTCCACCATTTGCATGAA | |
| | | | | GATACAATCTTCTATTATAG | |
| | | | | GCTAGGGTTAAGACAGTTAG | |
| | | | | GATTTTTGTATGAAGCTAAA | |
| | | | | CCCACTGAAGGAGTAAATAC | |
| | | | | ATTTAATAGTGACTTTAGAT | |
| | | | | TAATTAGAAAAGAACTCATT | |
| | | | | TACAAGATAACGATCATCTA | |
| | | | | T | |

TABLE 3
Polymorphisms Somewhat Associated with Autoflowering Phenotype in Cannabis,
Located within +/−500 kb of UPF2 gene

| | | | Predictive | | SEQ |
|---|---|---|---|---|---|
| | | | Variant | | |
| Polymorphism | | | of Auto- | | ID |
| ID | Chromosome | Haplotype | flowering | Genomic Context Sequence | NO: |
| SCM010796.2_ | CM010796.2 | C/G | G | CATttggttattatatatatattcatgatttttttttttctattt | 25 |
| 1001 | | | | attactATTGACAATGttgttattttagttatttcaaacttttaa | |
| | | | | ttgcattacaCttgtaaggataatatttttatggagtattaaata | |
| | | | | taaataaagagtgatatttaatttttatgtatttttcttcatatt | |
| | | | | tggaaattaatactaaaaatt | |
| SCM010796.2_ | CM010796.2 | T/C | C | ACTTGTTATAAATTATAGTTGAACTTCTTTATATTGTTATTGCTC | 26 |
| 1002 | | | | AAATAGAAGTTAACAATTATACAAGTCTTATACATTATGGGTTCA | |
| | | | | GAAACTTTGATTGAAGTGATATTGGCTATACTCCTTCCTCCAGTG | |
| | | | | GGTGTTTTCCTCCGCTTTGGTTGTGCGGtaataattaatcttaaa | |
| | | | | tatCTTATTATGTCTATGCCA | |
| SCM010796.2_ | CM010796.2 | G/T | T | CAAGGTCTGCTCAATTCCTTGATCATCAAGCAATCTCAATTCCTT | 27 |
| 1003 | | | | GATCATCAAGCAATTTATCAGTAGTGTTGTTAGTGGGTTTGCTAT | |
| | | | | TAGctttttgGtaaaaaaaaaaaaaaaaaaaaaaaaaaaacgagt | |
| | | | | gTTCGAGTCTCCTAATTTGAGCCAAAAAACACGAGACCGTTGTTG | |
| | | | | GCAATAATCCTCTTCTTAAGC | |
| SCM010796.2_ | CM010796.2 | C/T | T | aagaaggaggtttcggcatcaaagattcagagaaagagatccagg | 28 |
| 1004 | | | | ttcagatattgataatgctctgctacagaaaggaatcaagggcta | |
| | | | | gatatctgaaCggaaggagtcattatattccgctgcaaccaatgt | |
| | | | | aaggtttcctaaactttatatgtgtttatttcatcgttttagaaa | |
| | | | | gttcatatttagggtgttaat | |
| SCM010796.2_ | CM010796.2 | TAT/ | TT | TTTTTCTCCCATCTGATTCAAATGTAATATTTTGATTGAAATTCA | 29 |
| 1005 | | TT | | CTCAAATAAGAGGAAGACAGAGAAGGAGTGAAAAGACCAAGATTG | |
| | | | | GCAGAGTTATTATACCAAACAAGAAAAGAGCAAAATACTAACATG | |
| | | | | TAAGGATATTAAGACAAAGACAATTTAGTGATGAAACTTACAGAA | |
| | | | | GCTCTGAACTTATCAATCTCT | |
| SCM010796.2_ | CM010796.2 | T/C | C | TTGACACAACACGAAACTGGCACGAGCACAATTAGACACGAAAAA | 30 |
| 1006 | | | | ATATGGGCATTAGCACGACATAGGCACGACAAAAGATGAAAAATA | |
| | | | | TGTCCTTAAGTACAATACGACACGAGAAGTAGGGCTGAACATTTA | |
| | | | | AACCCGCAAACCCGAAAACCCGGCAACCCACCCCGACCCGCTGCC | |
| | | | | gaaaaaaaaccgaaaaattga | |
| SCM010796.2_ | CM010796.2 | G/A | A | ACCTTTGAAAGGGATCCTTTCATTTTGAACATGAACTTATTGTCT | 31 |
| 1007 | | | | CTTATACCGTCaagaatttaattaattctCCATAATAAAAAAGTA | |
| | | | | TTGAATGAGTGGTTTGGTGGTAAAATGCTTACTCTCAACTAGTTT | |
| | | | | TTTAAGCcgttaaatataagaaaaatgtcTCGCAGCTTAGCCACC | |
| | | | | CTAGACATCCATTTTAGGCAC | |
| SCM010796.2_ | CM010796.2 | T/A | T | Tttgattgtttttttaaaataagataaaaagataaaatagaaata | 32 |
| 1008 | | | | tttatgaaaataaagaattatataAGTGAAAATGTTTTAGACAAa | |
| | | | | acgtaaaaatTaaaaaaaaaaaaaaaaaaaaaaaaaaaaaaaaaa | |
| | | | | ggagtacAACGGTTTATGCAAACGtgtataattaacctaataatt | |
| | | | | ataataagaatAGAATTAGGa | |
| SCM010796.2_ | CM010796.2 | T/G | G | AAGAAGTTGAACCTCCTCATGAGACAAAGACAAATAGTAGTTCTT | 33 |
| 1009 | | | | TGACTGATCTTCAAAGAAGCATTATAAAAGAATTATTAGAAATCA | |
| | | | | TATCGTCTCCTATTGAAAATAAACAACAACAAGATCAATCTAATG | |
| | | | | AAgttgataaaataattattcaagaTGATGGCATAGGCCTCGATG | |
| | | | | ATCTCCCTGAGGAATTCTTCA | |
| SCM010796.2_ | CM010796.2 | A/C | C | attttttatgaatcttAAATTCAAAttctttttttaatgttttat | 34 |
| 1010 | | | | atggttttttttttttttttagtttaattggttaaataatgtcat | |
| | | | | atttagtggcAtttactttttatgtcatatttatcataaccttaa | |
| | | | | taattttttccatattttttaaaatagccCATCAAATTTTGTCCC | |
| | | | | CGAATGTTAAGCGGGGCGGGA | |

TABLE 4
Polymorphisms Significantly Associated with Autoflowering
Phenotype, Located on Chromosome CM010796.2

| | | | Predictive | | SEQ |
|---|---|---|---|---|---|
| Polymorphism | | | Variant of | | ID |
| ID | Chromosome | Haplotype | Autoflowering | Genomic Sequence Context | NO: |
| SCM010796.2_ | CM010796.2 | G/A | A | aataataaataaaaactttaacaaaaaaaattaattaaaaaatta | 35 |
| 2001 | | | | ttcgaactttaaattaattttaataaactaACCAATTTCATGAGT | |
| | | | | TACTAATaatGttttattaatttatatttaaaagttaatctTGTA | |
| | | | | TCTTTGCTAAACTTGTATTATAGAGCAATTTTaatagatttttaa | |
| | | | | aattatttataaaagtattaa | |
| SCM010796.2_ | CM010796.2 | T/C | C | aaataattattatttagttttGATAATAAAGTTATTGGTaagatt | 36 |
| 2002 | | | | tcatatatatacatattattaataactttttatatttgattataa | |
| | | | | tttattaattTcttaaatattaattaacatgaaaaaattactaaa | |
| | | | | taaagTAACATAAATATATCTTAAATTAGCAATCAATTTCATGGG | |
| | | | | ttacttaaaattatttttatt | |
| SCM010796.2_ | CM010796.2 | A/T | T | taattaaatttcttaatttgTAACTAATCTATAAGAagtcatgaa | 37 |
| 2003 | | | | aagaaaaaatattttaagaaaattggTAAGCAAATTTATAattca | |
| | | | | tataataataAtagattgATGTAAATTATAGAGAAAAGTAAAACT | |
| | | | | CAATACCAAATTACAATAAATCTTAAATTATAAATGTTAGATCGT | |
| | | | | AAGTagaccaaacaaaaaaaa | |
| SCM010796.2_ | CM010796.2 | C/T | T | acataaattaattaataactataTACTGATTGAATAAAGAGGATA | 38 |
| 2004 | | | | AGAAAAAACTTATCACATCAAAATGATCTCCAAatCttcattatt | |
| | | | | tttaaatatcCcATTTGTTTCGAACATTGTTATATATCTACTTTG | |
| | | | | TTCCACCTACACCACTTAGACGAAACCCAATGCAGTTTGCTAACG | |
| | | | | TTAAAGATTAGCCAATTTAGT | |
| SCM010796.2_ | CM010796.2 | T/A | A | TGATTGAATAAAGAGGATAAGAAAAAACTTATCACATCAAAATGA | 39 |
| 2005 | | | | TCTCCAAatcttcattatttttaaatatcccATTTGTTTCGAACA | |
| | | | | TTGTTATATATCTACTTTGTTCCACCTACACCACTTAGACGAAAC | |
| | | | | CCAATGCAGTTTGCTAACGTTAAAGATTAGCCAATTTAGTTTACA | |
| | | | | TTTTCATTACATTTTGCTCTA | |
| SCM010796.2_ | CM010796.2 | A/G | G | TGATTATTCGGATTGAAACAatagataatttatagcgtatctatt | 40 |
| 2006 | | | | cTTGGTGAATAGAGTATTTTATGTAATCAGGAGTGCAATTTCGAA | |
| | | | | TCTATAGTTGAGTGAGGAggaattaataataaagaaaatttactt | |
| | | | | gataaattctaGAATTACTTATTGAgtgcttgattatataggccc | |
| | | | | atgtccttgtactagttgaga | |
| SCM010796.2_ | CM010796.2 | G/A | A | ggaattaataataaagaaaatttacttgataaattctaGAATTAC | 41 |
| 2007 | | | | TTATTGAgtgcttgattatataggcccatgtccttgtactagttg | |
| | | | | agataataatGtcttgtagactcaattaattaattttaattaatc | |
| | | | | aattagaattctaTTTATGagtttcactaagtaagggcttatttg | |
| | | | | agaagaaaatgaggatttaag | |
| SCM010796.2_ | CM010796.2 | T/C | C | AAGGTACAATCTATGctactatattttaaaatattcattATACAT | 42 |
| 2008 | | | | TGTAAATATTGaaatatgtaaattttttacTTATATTTCAGGTTG | |
| | | | | AaagtatataTgtttatatatgcaCACACGTCTATAATGAAAggt | |
| | | | | ttttgttttaatttcaatttcaagaataaaaattacagtaattta | |
| | | | | aattgaattcatatctttctt | |
| SCM010796.2_ | CM010796.2 | T/C | C | ATCTATGctactatattttaaaatattcattATACATTGTAAATA | 43 |
| 2009 | | | | TTGaaatatgtaaattttttacTTATATTTCAGGTTGAaagtata | |
| | | | | tatgtttataTatgcaCACACGTCTATAATGAAAggtttttgttt | |
| | | | | taatttcaatttcaagaataaaaattacagtaatttaaattgaat | |
| | | | | tcatatctttcttttAATTAC | |
| SCM010796.2_ | CM010796.2 | C/T | T | catattttaatagtggtatatataattttgttagcatgatatata | 44 |
| 2010 | | | | ttttttgagtattaatttagtattgttataatttttttgtggtat | |
| | | | | ataattttatCactatagtatattaattttcagtaATACGATATA | |
| | | | | TCAAATACTGctgtatatatttaatgatctaatattttattcatt | |
| | | | | ttgttacaatataataatatg | |

1. A method for identifying whether or not a Cannabis plant has a day length neutral phenotype, or is a carrier of a trait for the day length neutral phenotype, the method comprising testing nucleic acid from the Cannabis plant to determine the presence or absence of a variation at a polymorphic site in a region consisting of an endogenous UPF2 gene within an about 20 megabase region in Cannabis chromosome CM010796.2 located between about 40 megabases to about 60 megabases, wherein the presence of the variation at the polymorphic site indicates that the plant has the day length neutral phenotype or is a carrier of the trait for the day length neutral phenotype.

2-4. (canceled)

5. The method of claim 1, wherein the variation is one or more of a loss of function mutation, a gain of function mutation, a point mutation, a deletion, an insertion, a substitution, and a nonsense mutation.

6-13. (canceled)

14. The method of claim 1, wherein the variation is a deletion is in the 3′ untranslated region of the endogenous UPF2 gene.

15. The method of claim 14, wherein the deletion comprises a portion of SEQ ID NO: 2.

16. The method of claim 15, wherein testing nucleic acid from the Cannabis plant to determine the presence or absence of the variation in the polymorphic site comprises testing for the presence or absence of SEQ ID NO: 2, or a portion thereof wherein the absence of SEQ ID NO: 2 or a portion thereof in both copies of the endogenous UPF2 gene indicates that the plant has a day length neutral phenotype, and wherein the absence of SEQ ID NO: 2 or a portion thereof from only one copy of the UPF2 gene indicates that the plant is a carrier of the trait for the day length neutral phenotype.

17. The method of claim 1, wherein the presence of the variation in both copies of the chromosome indicates that the plant has a day length neutral phenotype.

18. The method of claim 1, wherein the presence of the variation in only a single copy of the chromosome indicates that the plant is a carrier of the trait for the day length neutral phenotype.

19. A method for identifying whether a Cannabis plant has a day length neutral phenotype, or is a carrier of a trait for a day length neutral phenotype, the method comprising testing nucleic acid from the Cannabis plant to determine the presence or absence of an allele that is in linkage disequilibrium r²=0.9 to 1 with a variation in a polymorphic site in a region consisting of an endogenous UPF2 gene within an about 20 megabase region in Cannabis chromosome CM010796.2 located between about 40 megabases to about 60 megabases, wherein the presence of the allele indicates that the plant has a day length neutral phenotype or is a carrier of the trait for the day length neutral phenotype.

20-22. (canceled)

23. The method of claim 19, wherein the presence of the variation in the polymorphic site indicates that the plant has a day length neutral phenotype or is a carrier of the trait for the day length neutral phenotype.

24-38. (canceled)

39. The method of claim 1, wherein said testing is carried out using an allele-specific primer that comprises a sequence selected from the group consisting of SEQ ID NOS: 3, 4 and sequences fully complementary thereto.

40-51. (canceled)

52. A method for producing a day length neutral Cannabis plant, the method comprising decreasing the expression of an endogenous UPF2 gene within an about 20 megabase region in Cannabis chromosome CM010796.2 located between about 40 megabases to about 60 megabases in the plant.

53-54. (canceled)

55. The method of claim 52, wherein decreasing the expression of the one or more endogenous genes comprises introducing or producing a loss of function allele in the endogenous gene.

56. The method of claim 55, where the loss of function allele or the gain of function allele comprises one or more of a point mutation, an insertion, a deletion, a substitution, and a nonsense mutation.

57-59. (canceled)

60. The method of claim 56, wherein the insertion is a T-DNA or a transposable element.

61. The method of claim 52, wherein decreasing the expression of the one or more endogenous genes comprises expressing a heterologous nucleic acid molecular homologous to a portion of the one or more endogenous genes, wherein the heterologous nucleic acid molecule decreases expression of the one or more endogenous genes.

62. The method of claim 61, wherein the heterologous nucleic acid molecule decreases expression of the one or more endogenous genes by RNA interference.

63. (canceled)

64. The method of claim 61, wherein the heterologous nucleic acid molecule comprises a portion of SEQ ID NO: 1.

65-70. (canceled)

71. A genetically modified Cannabis plant or plant cell having a day length neutral phenotype, wherein the Cannabis plant or plant cell is genetically modified to have reduced expression of an endogenous UPF2 gene within an about 20 megabase region in Cannabis chromosome CM010796.2 located between about 40 megabases to about 60 megabases.

72. (canceled)

73. The plant or plant cell of claim 71 comprising an expression construct for reducing the expression of the one or more endogenous genes.

74. The plant or plant cell of claim 73, wherein the expression construct comprises a nucleic acid molecule encoding a hairpin RNA for reducing expression of the endogenous UPF2 gene.

75. (canceled)

76. The plant or plant cell of claim 71, wherein the nucleic acid molecule encoding the hairpin RNA comprises a portion of SEQ ID NO: 1.

77. The plant or plant cell of claim 71, wherein the plant or plant cell is a seed or seed cell.

78. Seed produced by the Cannabis plant or plant cell as defined in claim 71.

79. Plant material of the Cannabis plant or plant cell as defined in claim 71.

80. Dried flower comprising the Cannabis plant cell as defined in claim 71.

81-89. (canceled)

90. A cannabinoid extracted or isolated from dried flower as defined in claim 80.

91. (canceled)

92. A terpene extracted or isolated from dried flower as defined in claim 80.

93. An extract of a Cannabis plant or plant cell as defined in claim 71, wherein the extract comprises one or more cannabinoids and/or one or more terpenes.

94. A concentrate produced from a Cannabis plant or plant cell as defined in claim 71, wherein the concentrate comprises one or more cannabinoids and/or one or more terpenes.

95. An isolate from a Cannabis plant or plant cell as defined in claim 71, wherein the isolate comprises one or more cannabinoids and/or one or more terpenes.

96. An oil isolated from a Cannabis plant or plant cell as defined in claim 71, wherein the oil comprises cannabinoids and/or terpenes.

97-142. (canceled)
